SEQUENCE LISTING (36 sequences)

SEQ ID NO: 1 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 2 - protein, 112 aa, Oryctolagus cuniculus
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 3 - protein, 12 aa, Oryctolagus cuniculus
TLSSAHKTDTID

SEQ ID NO: 4 - protein, 7 aa, Oryctolagus cuniculus
GSYTKRP

SEQ ID NO: 5 - protein, 9 aa, Oryctolagus cuniculus
GADYIGGYV

SEQ ID NO: 6 - protein, 121 aa, Oryctolagus cuniculus
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 7 - protein, 5 aa, Oryctolagus cuniculus
AYYMS

SEQ ID NO: 8 - protein, 17 aa, Oryctolagus cuniculus
TIYPSSGKTYYATWVNG

SEQ ID NO: 9 - protein, 12 aa, Oryctolagus cuniculus
DSYADDGALFNI

SEQ ID NO: 10 - protein, 125 aa, Mus musculus
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 11 - protein, 109 aa, Mus musculus
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 12 - protein, 10 aa, Mus musculus
GFTFNKYAMN

SEQ ID NO: 13 - protein, 19 aa, Mus musculus
RIRSKYNNYATYYADSVKD

SEQ ID NO: 14 - protein, 14 aa, Mus musculus
HGNFGNSYISYWAY

SEQ ID NO: 15 - protein, 14 aa, Mus musculus
GSSTGAVTSGYYPN

SEQ ID NO: 16 - protein, 7 aa, Mus musculus
GTKFLAP

SEQ ID NO: 17 - protein, 9 aa, Mus musculus
ALWYSNRWV

SEQ ID NO: 18 - protein, 15 aa, Homo sapiens
WNISSELNKDSYLTL

SEQ ID NO: 19 - protein, 10 aa, Artificial Sequence
  artificial linker sequence
GGGGSGGGGS

SEQ ID NO: 20 - protein, 14 aa, Homo sapiens
KSQKPYKIDSKQAS

SEQ ID NO: 21 - protein, 125 aa, Mus musculus
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 22 - protein, 109 aa, Mus musculus
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 23 - protein, 5 aa, Mus musculus
TYAMN

SEQ ID NO: 24 - protein, 19 aa, Mus musculus
RIRSKYNNYATYYADSVKG

SEQ ID NO: 25 - protein, 14 aa, Mus musculus
HGNFGNSYVSWFAY

SEQ ID NO: 26 - protein, 14 aa, Mus musculus
GSSTGAVTTSNYAN

SEQ ID NO: 27 - protein, 7 aa, Mus musculus
GTNKRAP

SEQ ID NO: 28 - protein, 9 aa, Mus musculus
ALWYSNLWV

SEQ ID NO: 29 - protein, 451 aa, Mus musculus
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 236 aa, Mus musculus
MGWSCIILFLVATATGVHSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 694 aa, Mus musculus
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 451 aa, Mus musculus
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 214 aa, Mus musculus
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 34 - protein, 467 aa, Mus musculus
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 702 aa, Mus musculus
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 246 aa, Homo sapiens
MGWSCIILFLVATATGVHSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2017/0306044 A1
(43) Pub. Date: Oct. 26, 2017

(54) BISPECIFIC ANTIBODIES AGAINST CD3EPSILON AND ROR1 FOR USE IN THE TREATMENT OF OVARIAN CANCER

(71) Applicant: ENGMAB AG, Wilen (CH)

(72) Inventors: MINH DIEM VU, WOLLERAU (CH); KLAUS STREIN, WEINBEIM (DE); OLIVER AST, BASSERSDORF (CH); TANJA FAUTI, ZUERICH (CH); ANNE FREIMOSER-GRUNDSCHOBER, ZUERICH (CH); RALF HOSSE, CHAM (CH); CHRISTIAN KLEIN, BONSTETTEN (CH); EKKEHARD MOESSNER, KREUZLINGEN (CH); SAMUEL MOSER, ROTKREUZ (CH); RAMONA MURR, ZUERICH (CH); PABLO UMANA, WOLLERAU (CH); SABINE JUNG-IMHOF, PLANEGG (DE); STEFAN kLOSTERMANN, NEURIED (DE); MICHAEL MOLHOJ, MUENCHEN (DE); JOERG REGULA, MUENCHEN (DE); WOLFGANG SCHAEFER, MANNHEIM (DE)

(21) Appl. No.: 15/517,329

(22) PCT Filed: Oct. 8, 2015
(86) PCT No.: PCT/EP2015/073309

§ 371 Date: Apr. 6, 2017

(30) Foreign Application Priority Data
Oct. 9, 2014 (EP) 14188378.5
Oct. 14, 2014 (EP) 14188727.3
Oct. 14, 2014 (EP) 14188728.1

(51) Int. Cl. C07K 16/30; C07K 16/46; C07K 16/28; C07K 16/40

(52) U.S. Cl. CPC C07K 16/3069; C07K 16/40; C07K 16/468; C12Y 207/10001; C07K 16/2803; C07K 2317/55; C07K 2317/54; C07K 2317/526; C07K 2317/524; C07K 2317/52; C07K 2317/35; C07K 2317/31; C07K 2317/56; C07K 2317/66; C07K 2317/73; C07K 2317/92; C07K 2317/94

(57) ABSTRACT

Bispecific antibodies against CD3epsilon and ROR1 are useful for use in the treatmentof ovarian cancer.

### BACKGROUND OF THE INVENTION

ROR1 (synonyms: tyrosine-protein kinase transmembrane receptor ROR1, EC=2.7.10.1, neurotrophic tyrosine kinase, receptor-related 1, UniProtKB Q01973) is a tyrosine-protein kinase receptor. The receptor is described in Masiakowski P., Carroll R.D., J. Biol. Chem. 267:26181-26190(1992) “A novel family of cell surface receptors with tyrosine kinase-like domain.” WO9218149 and WO9527060 mention ROR-1 as Rtk-2 and antibodies against ROR-1. WO2002087618 mentions a method of controlling the growth and differentiation of cancer by selectively inhibiting a growth factor receptor. Such a receptor would be Ror1 or Ror2. WO2005100605 mentions ROR1 as a therapeutic target for breast cancer and anti ROR1 antibodies which specifically bind to ROR1, to the extracellular region of ROR1 (MI -V406) and. ROR1 fragments Q73-V139, E165-I299, K312-C391. WO2007051077 relates to an anti-ROR1 antibody and its use in lymphoma cell detection. WO2008103849 also mentions anti-ROR1 antibodies. Rabbani (Blood (ASH Annual Meeting Abstracts) 21)10 116: Abstract 916) discloses the use of anti ROR1 antibodies for the treatment of chronic Lymphocytic leukemia (CLL). Rabbani used anti-ROR1 an antibody against the extracellular domain, an antibody against the CRD region (ligand binding site for Wnt proteins) and an antibody against the kringle domain. Darieshmanesh AH et al., hit. J. Cancer, 123 (2008) 1190-1195 relates to an anti ROR1 antibody that binds to the extracellular domain fragment WNISSELNKDSYLTL (SEQ ID NO:18) and an anti ROR1 antibody that binds to the intracellular domain fragment KSQKPYKIDSKQAS (SEQ ID NO:20). Also the use of such antibodies for the treatment of CLL is mentioned.

Zhang H. et al., SCIENTIFIC REPORTS 4 : 58H DOI: 10.1038/srep0581.1 (24 July 2014) reports that ROR1 protein expression is correlated with poor clinical outcome in human ovarian cancer.

WO2011159847 relates to an anti-ROR1 antibody as a conjugate with a biologically active molecule for the treatment of ROR1 cancer like lymphoma or adenocarcinoma. WO2008076868, WO2008103849, WO201008069, WO2010124188, WO2011079902, WO2011054007, WO2011159847, WO2012076066, WO2012076727, WO 2012045085, and WO2012097313 relate also to ROR1 binding molecules or anti ROR1 antibodies. WO2012075158 relates to an anti-ROR1 antibody comprising as light chain variable domain (VL) the sequence of SEQ NO:2 and as variable heavy chain domain (NTH) the sequence of SEQ ID NO:6, and as respective CDRs the sequences of SEQ ID NO: 3, 4, 5, 7, 8, 9. This antibody is further named as MAB 1.

WO2005040413 is directed to a screening method for the identification and/or validation of inhibitors of a receptor tyrosine kinase activity, including ROR1.

WO2008036449, WO2011014659 and WO2011050262 mention bispecific antibodies wherein one target can be ROR1. WO2007146968 mention multivalent single-chain binding proteins with effector function and ROR1 and CD3 are mentioned as possible targets. WO2011054007 is directed to a method of treatment of cancer administering an affinity reagent which binds to the extracellular domain of ROR1. Bispecific antibodies with CD3 are also mentioned. WO2014031174 mentions bispecific antibodies which are specific to two different epitopes of ROR1.The preferred antibody DIO strongly internalizes at 37° C. in MDA MB 231 epithelial breast adenocarcinoma. Yang and Baskar PLos ONE 6 (2011) e21018, like WO2012075158, mention also anti-ROR1 antibody R12. Rebagay R. et al., Frontiers in Oncology (2012) 7, Article 34, 1-8 mention that RORs are pharmaceutical targets and a means to deliver cytotoxic agents in the cells which express the target on the cell surface. Rebagay also mention bispecific antibodies such as BiTE. Strong internalization is favorable for armed antibodies i.e. antibody drug conjugates according to Rebagay. D. MEZZANZANICA ET AL, INTERNATIONAL JOURNAL OF CANCER, 41 (1988) 609-615 investigated a therapeutic approach by retargeting CTLs by a bispecific antibody consisting of 1MOv 18 (a poorly internalizing antibody specific for human ovarian carcinoma cells) and an anti-CD3 antibody (OKT3 or TR.66). M. HUDECEK ET AL., BLOOD, 116 (2010), 4532-4541, mention that ROR1 is expressed by B cell chronic lymphocytic leukemia (B-CLL) and mantle cell lymphoma (MCL). Such cells can be targeted by activated CD8 T cells transfected with, and expressing scFv from murine anti-ROR1 antibody 2A2. Such cells are useful for treatment of B cell malignancies. Baskar S. et al., mAbs 4.3 (2012) 349-361 relate to the targeting of malignant B cells with an immunotoxin BT-1 comprising scFv 2A2 anti-ROR1 conjugated to PE38 toxin. The immunotoxin is partially internalized and induces apoptosis. PCT/EP2014/057199 relates to bispecific antibodies against CD3 and ROR1. EP14188378 relates to charge variants of bispecific antibodies against CD3 and ROR1.

The TCR/CD3 complex of T-Lymphocytes consists of either a TCR alpha (α)/beta (β) or TCR gamma (γ)/delta (δ) heterodimer coexpressed at the cell surface with the invariant subunits of CD3 labeled gamma (γ), delta (δ), epsilon (ε), zeta (ζ), and eta (η) Human CD3ε is described under UniProt P07766 (CD3ε_HUMAN). An anti CD3ε antibody described in the state of the art is SP34 (Yang S J, The Journal of immunology (1986) 137; 1097-1100). SP34 reacts with both primate and human CD3. SP34 is available from PharMingen®. A further anti CD3 antibody described in the state of the art is UCHT-1 (see WO2000041474). A further anti CD3 antibody described in the state of the art is BC-3 (Fred Hutchinson Cancer Research Institute; used in Phase I/II trials of GvHD, Anasetti et al,, Transplantation 54: 844 (1992)).

A wide variety of recombinant bispecific antibody formats have been developed in the recent past, e.g. by fusion of, e.g. an IgG antibody format and single chain domains (see Kontermann R E, mAbs 4:2, (2012) 1-16). Bispecific antibodies wherein the variable domains VL and VH or the constant domains CL and CH1 are replaced by each other are described in WO2009080251 and WO2009080252.

An approach to circumvent the problem of mispaired byproducts, which is known as ‘knobs-into-holes’, aims at forcing the pairing of two different antibody heavy chains by introducing mutations into the CH3 domains to modify the contact interface (Ridgway J B, Presta L G, Carter P; and WO1.9960270 1. 1, Merchant AMT, et al, Nature Biotech 16 (1998) 677-681; S, Ridgway J B, Wells J A, Carter P., J Mol Biol 270 (1997) 26-35, EP 1870459A1, Xie, Z., et al, J Itnmunol Methods 286 (2005) 95-101, WO2012116927, WO2010145792, WO2009080254. WO 2006093794 relates to heterodimeric protein binding compositions. WO199937791 describes multipurpose antibody derivatives. Morrison, S.L., et al., J. Immunol. 160 (1998) 2802-2808 refers to the influence of variable region domain exchange on the functional properties of ligG.

WO 201302362 relate to heterodimerized polypeptides. WO201312733 relates to polypeptides comprising heterodimeric Fc regions. WO2012131555 relates to engineered heterodimeric immunoglobulins. EP 2647707 relates to engineered hetero-dimeric immunoglobulins. WO2009080251, WO 2009080252, WO 2009080253, WO 2009080254 and Schaefer, W. et al, PNAS, 108 (2011) 11187-1191 relate to bivalent, hispecific IgG antibodies with a domain crossover.

Ovarian cancer is the leading cause of death from gynecologic cancer in the United States and the seventh most common cancer and the eighth most common cause of death from cancers in women. An estimated 21,980 new cases of ovarian cancer and 14,270 deaths related to ovarian cancers are expected in the United States in 2014. Worldwide, nearly 225,000 women will be diagnosed with ovarian cancer, and more than 140,000 will die of the disease (Cancer Facts & FIGS. 2014; http://www.cancer.org), The incidence of ovarian cancer increases with age and is most prevalent in the eighth decade of life. About half of the women diagnosed with ovarian cancer are 63 years or older.. Ovarian cancer usually has a relatively poor prognosis. If diagnosed at the localized stage, the 5-year survival rate is 92%, however, only 15% of all cases arc detected at this stage. The majority of cases (61%) are diagnosed after the disease has already metastasized. For women diagnosed with distant metastases, the 5-year survival rate is 27%. Despite advances in surgery and chemotherapy over the past two decades, only modest progress has been achieved in improving the overall survival in patients with ovarian cancer. Although the majority of women with advanced ovarian cancer respond to first-line chemotherapy, most responses are not durable. More than 80% of patients will have a recurrence of their disease after first-line treatment, and more than 50% will die of recurrent disease within 5 years of diagnosis (http://www.cancerresearch.org). Targeted therapy is a newer type of cancer treatment that uses drugs or other substances to identify and attack cancer cells while doing little damage to normal cells. The targeted therapy drug that has been studied the most in ovarian cancer is bevacizumab (Avastink). In studies, beva.cizumab has been shown to shrink or slow the growth of advanced ovarian cancers. Trials to see if bevacizumab works even better when given along with chemotherapy have shown good results in terms of shrinking (or stopping the growth of) tumors, but it has not yet been shown to help women live longer (http://www.cancer.orgicancerlovariancancer).

Accordingly, there is a need for a further approach for the treatment of ovarian cancer.

### SUMMARY OF THE INVENTION

The invention relates to a bispecific antibody specifically binding to the two targets human CD3ε (further named also as “CD3”) and the extracellular domain of human ROR1 (further named also as “ROR1”) for use in the treatment of ovarian cancer. The treatment is performed in a patient suffering from ovarian cancer.

The invention relates to a the use of a bispecific antibody specifically binding to the two targets human CD 3r (further named also as “CD3”) and the extracellular domain of human ROR1 (further named also as “ROR1”) for the treatment of ovarian cancer in a patient suffering from ovarian cancer.

The invention relates to a method of treating ovarian cancer in a patient suffering from ovarian cancer comprising administering a therapeutically effective amount of a ^{.}bispecific antibody specifically binding to the two targets human CD3ε (further named also as “CDS”) and the extracellular domain of human ROR1 (further named also as “ROR1”).

Preferably the bispecific antibody used according to the invention is characterized in consisting of one Fab fragment of an anti-CD3 antibody (CD3 Fab), one or two Fab fragments of an anti-ROR.1 antibody (ROR1 Fab) and no or one Fc fragment. Preferably the bispecific antibody used according to the invention is characterized in comprising a monovalent anti-ROR1 antibody specifically binding to ROR1, and a monovalent antibody specifically binding to CD3. Preferably the bispecific antibody used according to the invention is characterized in being bivalent and comprising a monovalent anti-ROR1 antibody specifically binding to ROR1, and a monovalent antibody specifically binding to CD3. Preferably the bispecific antibody used according to the invention is characterized in being trivalent and comprising a bivalent anti-ROR1 antibody specifically binding to ROR1, and a monovalent Fab fragment of an antibody specifically binding to CD3

Preferably in the light chain and heavy chain of the CD3 Fab the variable domains VL and VH or the constant domains CL and CH1 are replaced by each other (CD3 crossFab). The CD3 Fab is N-terminally linked to the C-terminus to the ROR1 Fab. Preferably the VH domain of the CD3 Fab is N-terminally linked to the C-terminus of the CH1 domain of the ROR1 Fab. The Fc part is linked via its hinge region to the C-terminus of the respective Fab. Preferably the bispecific antibody used according to the invention is selected from the group of the constructs

a) CD3 Fab-ROR1 Fab,

b) CD3 Fab-ROR1 Fab-ROR1 Fab,

c) Fc-CD3 Fab-ROR1 Fab, and

d) ROR1 Fab Fc-CD3 Fab-ROR1 Fab.

The preferred constructs comprise as CD3 Fab a CD3 crossFab. The two ROR1 Fabs of constructs b) and d) are derived from the same anti-ROR1 antibody and comprise at least the same CDRs or the same VH, VL, CH1 and CL domains.

The preferred bispecific antibodies are shown in FIG. 1

The constructs are composed of the building blocks of SEQ ID NO: 30 to 36. The invention comprises therefore a polypeptide selected from the group consisting of the polypeptides of SEQ ID NO: 30, 31, 32, 33, 34, 35, and 36 the respective nucleic acids and their use for the preparation of the constructs.

The invention relates further to a construct selected from the group of

a) construct consisting of building blocks SEQ ID NO:30 (2×), 31, 32, and 33 (Fia.1A)

b) construct consisting of building blocks SEQ ID NO:30, 31, 33, and 36 (Fig.1B)

c) construct consisting of building blocks SEQ ID NO:30 (2×), 33, and 35 (Fig. C)

d) construct consisting of building blocks SEQ ID NO: 30, 33, and 34 (Fig.1D)

In a further embodiment the CD3 Mab sequences (VH and/or VL) within SEQ ID NO: 31, 33, 34, 35 are replaced by the respective VH and/or VL sequences of SEQ NO:21 and 22.

The invention relates to a bispecific antibody specifically binding to the two targets human CD3ε (further named also as “CD3”) and the extracellular domain of human ROM (further named also as “ROR1”), characterized in that the bispecific antibody does not internalize in a cell based assay at 37° C. during 2 hrs, using ROR1-positive primary B-CLL cells, and used at an antibody concentration of 1 nM, whereby not internalize means, that the mean fluorescence intensity (MFI), as detected by flow cytometry, of a bispecific antibody upon binding to ROR1-positive primary B-CLL cells measured at time 0 is not reduced more than 50%, preferably not more than 30% when re-measured after a 2hr-incubation at 37° C.

Alternatively the bispecific antibody can comprise instead of the Fabs single chains consisting of the same domains. In such a case the variable domains VL and VH or the constant domains CL and CH1 are not replaced by each other.

In a further preferred embodiment of the invention the bispecific antibody is a single chain antibody.

In a further preferred embodiment of the invention the bispecific antibody comprising two antibody variable domains on a single polypeptide chain, wherein a first portion of the bispecific antibody is capable of recruiting the activity of a human immune effector cell by specifically binding to an effector antigen located on the human immune effector cell, said first portion consisting of one antibody variable domain, and a second portion of the bispecific antibody is capable of specifically binding to ROR1. Preferably the second portion comprises one anti-ROR1 antibody variable domain. Preferably the second portion comprises two anti-ROR1 antibody variable domains. Preferably said first portion is specifically binding to human CD3ε.

Preferably the bispecific antibody used according to the invention is a bivalent antibody and characterized in comprising a monovalent anti-ROR1 antibody specifically binding to ROR1, and a monovalent antibody specifically binding to CD3. A bivalent antibody is preferred if its said mean fluorescence intensity (MFI), as detected by flow cytometry, upon binding to ROR1-positive cells measured at time 0 is not reduced more than 50%, preferably not more than 30% by internalization when re-measured after a 2hr-incubation at 37° C. Preferably the bispecific antibody used according to the invention is a bivalent antibody and characterized in comprising a monovalent anti-ROR1 antibody specifically binding to ROR1, and a monovalent antibody specifically binding to CD3. Preferably the monovalent antibody specifically binding to CD3 is a Fab fragment, preferably a CD3 crossFab. Such a bivalent antibody is preferred if its said mean fluorescence intensity (ME), as detected by flow cytometry, upon binding to ROR1-positive cells measured at time 0 is not reduced more than 50%, preferably not more than 30% by internalization when re-measured after a 2hr-incubation at 37° C. Preferably the bispecific antibody used according to the invention is a trivalent antibody and characterized in comprising a bivalent anti-ROR1 antibody specifically binding to ROR1and a monovalent antibody specifically binding to CD3. Preferably the monovalent antibody specifically binding to CD3 is a Fab fragment or preferably a CD3 crossFab. A trivalent antibody is preferred if its said mean fluorescence intensity (MFI), as detected by flow cytometry, upon binding to ROR1-positive cells measured at time 0 is not reduced more than 50%, preferably not more than 30% by internalization when re-measured after a 2hr-incubation at 37° C.

Preferably the bispecific antibody used according to the invention is characterized in that e bispecific antibody does not internalize in said cell based assay at 37° C. during 24 hrs.

Preferably the bispecific antibody used according the invention does not internalize in said cell based assay if used in a concentration between 0.1 pM and 200 nM.

A further embodiment of the invention is an antibody used according to this invention with an affinity ratio of ROR1 to CD3 of 5000:1 to 5:1, as determined by Kd values using surface plasmon resonance. Such an antibody is favorable because of its stronger binding to malignant cells over T cells. Preferably the Kd values are about 100 nM for the CD3 antibody and about 50 pM to 50 nM for the ROR1 antibody.

In a preferred embodiment of the invention the antibody used according to the invention consists of one Fab fragment of an antibody specifically binding to CD3 (further named also as “CD3-Fab”), and one Fab fragment of an antibody specifically binding to ROR1 (further named also as “ROR1-Fab(s)”) and a Fe part, wherein the CDS-Fab and the ROR1-Fab are linked via their C-termini to the hinge region of said Fe part (FIG. 1E).

In a preferred embodiment of the invention the antibody used according to the invention consists of one CD3-Fab, and one ROR1-Fab and an Fc part, wherein the CD3-Fab and the ROR1-Fab are linked via their C-termini to the hinge region of said Fc part and a second ROR1-Fab, which is linked with its C-terminus to the N-terminus of the CD3-Fab, The CD3-Fab comprises crossover (FIGS. 1A). Especially preferred is a bispecifie antibody comprising ROR1 -Fab-fc-CD3-Fab-ROR1 -Fab, and the CD3-Fab comprises CL/CH1 crossover (FIG. 1A). Especially preferred is that both ROR1 -Fabs comprise as CDRs the CDRs of antibody MAB1, or as VH/VL the VH/VL of MAB 1.

In a preferred embodiment of the invention the antibody used according to the invention consists of two ROR1-Fabs and an Fc part, wherein the ROR1 -Fabs are linked via their C-termini to the hinge region of said Fc part and a CD3-Fab, which is linked with its C-terminus to the N-tenninus of one ROR1-Fab. The CD3-Fab comprises crossover (FIG. 1F).

In a preferred embodiment of the invention the antibody used according to the invention consists of one CD3-Fab, which is linked via its C-terminus to the hinge region of said Fc part and a ROR1 -Fab, which is linked with its C-terminus to the N-terminus of the CD3-Fab. The CD3-Fab comprises crossover (FIG. 1B).

In a preferred embodiment of the invention the antibody used according to the invention consists of one ROR1-Fab, which is linked via its C-terminus to the hinge region of said Fc part and a CD3-Fab, which is linked with its C-terminus to the N-tenninus of the ROR1-Fab. The CD3-Fab comprises crossover (Figure IG).

The Fab fragments are chemically linked together by the use of an appropriate linker according to the state of the art. Appropriate linkers are described e.g. in US 20140242079. Preferably a (Gly4-Serl)2 (SEQ ID NO:19) linker is used (Desplancq DK et al., Protein Eng. 1994 Aug; 7(8):1027-33 and Mack M. et al., PNAS Jul. 18, 1995 vol. 92 no. 15 7021-7025). Linkage between two Fab fragments is performed between the heavy chains. Therefore the C-terminus of CH1 of a first Fab fragment is linked to the N-terinus of VH of the second Fab fragment (no crossover) or to VL (crossover). Linkage between a Fab fragment and the Fc part is performed as linkage between CH1 and CH2.

The first and a second Fab fragment of an antibody specifically binding to ROR1 are preferably derived from the same antibody and preferably identical in the CDR sequences, variable domain sequences VH and VL and/or the constant domain sequences CH1 and CL. Preferably the amino acid sequences of the first and a second Fab fragment of an antibody specifically binding to ROR1 are identical. Preferably the ROR1 antibody is an antibody comprising the CDR sequences of antibody MABL an antibody comprising the VH and VL sequences of antibody MAB 1, or an antibody comprising the VH, VL, CH1 , and CL sequences of antibody MAB 1.

Preferably the hispecific antibody comprises as Fab fragments and Fc part, not more than one Fab fragment of an anti-CD3 antibody, not more than two Fab fragments of an anti-ROR1 antibody and not more than one Fc part, preferably a human Fc part. Preferably the second Fab fragment of an anti-ROR1 antibody is linked via its C-terminus either to the N-terminus of the Fab fragment of an anti-CD3 antibody or to the hinge region of the Fc part. Preferably linkage is performed between CH1 of ROR1-Fab and VH of CDS-Fab (CL/CH1 crossover).

In a further embodiment of the invention the bispecific antibody according to the invention is

a) of construct ROR1 Fab Fe-CD3 Fab-ROR1 Fab,

b) comprises CL/CH1 crossover within the Fab fragment of the anti-CD3 antibody,

c) comprises a human IgG1Fc part,

d) comprises within the Fc part: substitution of Pro329 with glycine and substitutions of Leu234 by alanine and Leu235 by alanine.

Preferably the antibody portion specifically binding to human CD3, preferably the Fab fragment, is characterized in comprising a variable domain VH comprising the heavy chain CDRs of SEQ ID NO: 12, 13 and 14 as respectively heavy chain CDR1, CDR2 and CDR3 and a variable domain VL comprising the light chain CDRs of SEQ ID NO: 15, 16 and 17 as respectively light chain CDR1, CDR2 and. CDR3 of the anti-CD3ε antibody (CDR MAB CD3 H2C). Preferably the antibody portion specifically binding to human CD3 is characterized in that the variable domains are of SEQ ID NO:10 and 11 (VERT MAB CD3 H2C).

Preferably the antibody portion specifically binding to human CD3, preferably the Fab fragment, is characterized in comprising a variable domain VII comprising the heavy chain CDRs of SEQ ID NO: 23, 24 and 25 as respectively heavy chain CDR1, CDR2 and CDR3 and a variable domain VL comprising the light chain CDRs of SEQ ID NO: 26. 27 and 28 as respectively light chain CDR1, CDR2 and CDR3 of the anti-CD3F, antibody (CDR MAB CD3 CH2527). Preferably the antibody portion specifically binding to human CD3 is characterized in that the variable domains are of SEQ ID NO:21 and 22 (VHVL MAB CD3).

Preferably the antibody portion, preferably the Fab fragment, specifically binding to human ROR1 is characterized in comprising a variable domain VH comprising the heavy chain CDRs CDR1H of SEQ ID NO:7, a CDR2H of SEQ ID NO:8, a CDR3H of SEQ ID NO: 9 and comprising a variable domain VL comprising the light chain CDRs CDR1L of SEQ NO:3, a CDR2L of SEQ NO:4, a CDR3L of SEQ ID NO: 5 (CDR MAB1).

Preferably the antibody portion, preferably the Fab fragment, specifically binding to human ROR1 is characterized in comprising a VH of SEQ ID NO: 6 and a VL of SEQ ID NO: 2 (VHVL MA,B1).

The invention further relates to a nucleic acid set encoding a respective heavy and light chain set.

Preferably the bispecific antibody used according to the invention comprising constant heavy regions CH2/CH3 of IgG1 subclass is characterized in comprising the mutations L234A, L235A and P239G (numbering according to Kabat) to avoid FcR and Clq binding and minimizing ADCC/CDC. The advantage is that such an antibody of the invention mediates its tumor cell killing efficacy purely by the powerful mechanism of T-cell redirection/activation, Additional mechanisms of action like effects on complement system and on effector cells expressing FcR are avoided and the risk of side-effects is decreased.

Preferably the antibody used according to the invention comprises a heavy chain of an antibody consisting of (from N-to-C-terminus) VH(ROR1)-CH1(ROR1)-VH(CD3)-CL(CD3)-CH2-CH3 of SEQ ID NO: 37, as well as the respective encoding nucleic acids. These polypeptides and respective nucleic acids are useful for the production of a bispecific antibody used according to the invention.

The amino acid (aa) exchanges (further mentioned as “charge variants”) outside of the CDRs of the bispecific antibodies used according to the invention provide considerably improved production/purification without changing biological properties like binding to ROR1. By introduction of the aa exchanges (charge variants) light chain LC inispairing and the formation of side products in production is significantly reduced and therefore purification is facilitated.

The invention relates preferably to the use of a bispecific antibody specifically binding to the two targets human CD3ε and the extracellular domain of human ROR1. which does not internalize. The ^{.}bispecific antibody used according to the invention is preferably characterized in not internalizing in a concentration of 1 nM in primary B-CLL cells at 37° C. during two hours. The bispecific antibody used according to the invention is preferably characterized in that the bispecific antibody does not internalize in a cell based assay 25 at 37° C. during 2 hrs, using ROR1 -positive primary B-CLL cells and used at an antibody concentration of 1 nM, whereby not internalize means, that the mean fluorescence intensity (MFI), as detected by flow cytometry, of a bispecific antibody upon binding to ROR1-positive primary B-CLL cells measured at time 0 is not reduced more than 50%, preferably not more than 30% when re-measured after a 2 hr-incubation at 37° C.

Preferably the bispecific antibody used according to the invention is a bivalent antibody and characterized in comprising a monovalent anti-ROR1 antibody specifically binding to ROR1, and a monovalent antibody specifically binding to CD3. A bivalent antibody is preferred if its said mean fluorescence intensity (MFI), as detected by flow cytometiy, upon binding to ROR1-positive cells measured at time 0 is not reduced more than 50%, preferably not more than 30% by internalization when re-measured after a 2 hr-incubation at 37° C. Preferably the bispecific antibody used according to the invention is a bivalent antibody and characterized in comprising a monovalent anti-ROR1 antibody specifically binding to ROR1, and a monovalent antibody specifically binding to CD3. Preferably the monovalent antibody specifically binding to CD3 is a Fab fragment, preferably a CD3 crossFab. Such a bivalent antibody is preferred if its said mean fluorescence intensity (MFI), as detected by flow cytometry, upon binding to ROR1-positive cells measured at time 0 is not reduced more than 50%, preferably not more than 30% by internalization when re-measured after a 2hr-incubation at 37° C. Preferably the bispecific antibody used according to the invention is a trivalent antibody and characterized in comprising a bivalent anti-ROR1 antibody specifically binding to ROR1, and a monovalent antibody specifically binding to CD3. Preferably the monovalent antibody specifically binding to CD3 is a Fab fragment or preferably a CD3 crossFab, A trivalent antibody is preferred if its said mean fluorescence intensity (MFI), as detected by flow cytometry, upon binding to ROR1-positive cells measured at time 0 is not reduced more than 50%, preferably not more than 30% by internalization when re-measured after a 2 hr-incubation at 37′C.

Preferably the bispecific antibody used according to the invention does not internalize in said cell based assay at 37° C. during 24 hrs.

Preferably the bispecific antibody used according the invention does not internalize in said cell based assay if used in a concentration between 0.1 pM and 200 nM.

A further embodiment of the invention is an antibody used according to this invention with an affinity ratio of ROR1 to CD3 of 5000:1 to 5:1, as determined by Kd values using surface plasmon resonance. Such an antibody is favorable because of its stronger binding to malignant cells over cells. Preferably the Kd values are about 100 nM for the CDS antibody and about 50 pM to 50 nM for the ROR1 antibody.

Preferably the antibody portion specifically binding to CD3 is characterized in being humanized. Preferably the CD3 Mab according to the invention binds to the same epitope of CD3ε as antibody 1-12C (described in WO2008119567) and/or antibody CH2527 (described in WO2013026839) or is preferably antibody H2C or CH2527.

Preferably the antibody portion specifically binding to ROR1 is characterized in comprising a light chain variable domain (VL) comprising as respective variable light chain CDRs the CDRs of SEQ ID NO: 3, 4, 5 and a heavy chain variable domain (VH) comprising as respective variable heavy chain CDRs the CDRs of SEQ ID NO:7, 8, 9, Preferably the antibody portion specifically binding to ROR1 is characterized in comprising as light chain variable domain (VL) a sequence being at least 90% identical to the sequence of SEQ ID NO:2 and as variable heavy chain domain (VH) a sequence being at least 90% identical to the sequence of SEQ ID NO:6, Preferably the antibody portion specifically binding to ROR1 is characterized in comprising as light chain variable domain (VL) the sequence of SEQ ID NO:2 and as variable heavy chain domain (VH) the sequence of SEQ ID NO:6. Preferably the antibody portion specifically binding to ROR1 is characterized in being humanized. Preferably the ROR1 Mab used according to the invention binds to the same epitope of ROR1 as the Mab mentioned above.

A bispecific antibody used according to the invention is produced by transforming a host cell with one or more vectors comprising nucleic acid molecules encoding the respective antibodies or fragments, culturing the host cell under conditions that allow synthesis of said antibody molecule; and recovering said antibody molecule from said culture.

Preferably the method for the preparation of a bispecific antibody used according to the invention comprising the steps of

a) transforming a host cell with one or more vectors comprising nucleic acid molecules encoding the heavy and light chain set of an antibody useful according to the invention

b) culturing the host cell under conditions that allow synthesis of said antibody molecule; and

c) recovering said antibody molecule from said culture.

A further embodiment of the invention is a host cell comprising vectors comprising nucleic acid molecules encoding an antibody used according to the invention.

A further embodiment of the invention is a host cell comprising vectors comprising nucleic acid molecules encoding the light chain and heavy chain of an antibody specifically binding to the first target and vectors comprising nucleic acid molecules encoding the light chain and heavy chain of an antibody specifically binding to the second target, wherein the variable domains VL and VH are replaced by each other.

A further preferred embodiment of the invention is a pharmaceutical composition comprising such antibody and a pharmaceutically acceptable excipient.

A further preferred embodiment of the invention is a pharmaceutical composition comprising an antibody according to the invention for use as a medicament. A further preferred embodiment of the invention is an antibody according to the invention or a pharmaceutical composition comprising an antibody according to the invention for use as a medicament in the treatment of ROR1 -positive ovarian cancers. ROR1 is expressed on human ovarian cancers at the mRNA and protein levels (Zhang H. et al.. Scientific Reports|4 : 581|DOI: 10.1038/srep05811 (24 Jul. 2014). A further embodiment of the invention is an antibody according to the invention or a pharmaceutical composition comprising an antibody according to the invention for use as a medicament in the treatment of ovarian cancers expressing ROR1. A preferred embodiment of the invention is an antibody according to the invention or a pharmaceutical composition comprising an antibody according to the invention for use as a medic,ment in the treatment of ovarian cancers.

A further embodiment of the invention is the se of an antibody according to the invention or the pharmaceutical composition according to the invention for such treatments.

Preferably the antibody according to the invention or the pharmaceutical composition is administered once or twice a week preferably via subcutaneous administration (e.g. preferably in the dose range of 0.1 to 10 mg/m² once or twice a week). Due to superior cytotoxicity activities of the antibody according to the invention, it can be administered at a lower magnitude of clinical dose range as compared to conventional monospecific antibodies or conventional bispecific antibodies that are not T cell bispecifics (i.e. do not bind to CD3 on one ann). It is envisaged that for an antibody according to the invention subcutaneous administration is preferred in the clinical settings in the dose range of 0.1-10 mg/m²once or twice a week)). An antibody according to the invention is eliminated with a half-life of about several days which allows at least once or twice/week administration. Another advantage of the antibody according to the invention is a molecular weight (i.e. approximately 150-200 kDa) higher than the kidney filtration size limit (50-70 kDa). This molecular weight allows long elimination half-life and makes subcutaneous administrations once or twice a week possible.

Preferably an antibody according to the invention is characterized by showing tumor growth inhibition of more than 70%, preferably of more than 85%, preferably of close to 100% in a xenograft model with a ROR1 expressing ovarian tumor cell lines (for example PA-1, MCAS, EFO-21, COLO-704, SW-626), preferably PA-1 and/or COLO-704, at a dose of 1 mg/kg body weight (BW) administered intravenously (i.v.) or subcutaneously (s.c.) or intraperitoneal (i.p,) twice a week or once a week, preferably 0.5 mg/kg BW administered i.v, or i.p. or s,c, twice a week or once a week, preferably at 0.1 mg/kg BW administered i.v. or i.p. or s.c. twice a week or once a week, preferably at 0.05 mg/kg BW administered i.v. or i.p. or s.c. twice a week or once a week, preferably at 0.01 mg/kg BW administered i.v. or i.p. or s.c twice a week or once a week, preferably at 5μg/kg MN administered i.v. or i.p. or s,c, twice a week or once a week.

Preferably an antibody according to the invention is characterized by an elimination half-life in mice, preferably cynom.olgus monkeys of longer than 12 hours, preferably 3 days or longer.

Preferably an antibody according to the invention is characterized in showing an ECSO value for binding to ROR1-positive ovarian cancer cell lines (e.g. PA-1, MCAS, EFO-21, COLO-704, SW-626), preferably PA-1 and/or COLO-704, of 30 nM or lower, preferably an EC50 value of 1.5 nM and lower.

Preferably an antibody according to the invention is characterized by its capability to induce redirected killing of RORT expressing ovarian tumor cells (e.g. PA-1, MCAS, EFO-21, COLO-704, SW-626), preferably PA-1 and/or COLO-704, in the presence of human T cells with an ECSO lower than 10 nM, preferably 1 nM, preferably 0.05 nM, preferably 0.02 nM, preferably 0.002 nM and lower.

Preferably an antibody according to this invention is characterized in that said antibody stored in standard formulation buffer at 37° C. preferably at 40° C., for 10 days, preferably up to 2 weeks, preferably up to 4 weeks, does not result in more than 10% changes (A), preferably not more than 5(.'/i) changes (A), in high molecular weight (HMW) species and/or low molecular weight (LMW) species and/or monomer content as compared to the said antibody stored in the same formulation buffer at −80° C. for the same period of storage.

### DESCRIPTION OF THE FIGURES

FIG. 1A-G. Preferred bi specific antibodies comprising the Fab fragments (specific to CDS and ROR) as specified: (1A) Fab ROR1-Fc-Fab CD3-Fab ROR1; (1B) Fc-Fab ROR1-Fab CD3; (1C) Fab CD3-Fab ROR1-Fab ROR1; (1D) Fab CD3-Fab ROR1; (1D) Fab ROR1-Fc- Fab CD3; (1F) Fab ROR1-Fc-Fab ROR1-Fab CD3; (1G) Fc-Fab CD3-Fab ROR1. Preferably, the Fabs CD3 include a CH1-CL crossover to 15 reduce LC mispairing and side-products. Fab CD3 and Fab ROR1 are linked to each other with flexible linkers.

FIG. 2. Binding of ROR1 IgG (ROR1 Mab 1, open symbols) and anti-ROR1/anti-CD3 TCB antibodies (ROR1 Mab1-TCB, closed symbols) to ovarian cancer cell lines SK-OV-3 (A) and PA-1 (B) as measured by an increase in the median fluorescence intensity signal in function of antibody concentrations. No signal was observed with the control-TCB binding to CD3 only and not to ROR1 tested on both SK-OV-3 and PA-1 ovarian cancer cell lines (A and B; closed circles).

FIG. 3. Binding of anti-ROR1/anti-CD3 TCB antibodies to Jurkat T cells. A concentration-dependent binding of ROR1 Mab -TCB (squares) and control-TCB (circles) was observed on Jurkat T cells confirming that both TCB antibodies bind to CD3 on T cells.

FIG. 4. Up-regulation of T-cell activation markers by anti-ROR1/anti-CD3 TCB antibodies in presence of ovarian cancer target cells. The expression of activation markers was determined by measuring the median fluorescence intensity gated on CD4+ and CD8+ T cell populations. ROR1 Mab 1 -TCB (squares) induced a concentration-dependent increase of CD69 early activation marker which was observed on CD4+ T cells (A) and CD8+ T cells (B) in presence of ROR1-low expressing SK-OV-3 target cells while control-TCB (triangles) did not induce any T-cell activation. At a clinically relevant concentration of 1 nM of ROR1 Mab1-TCB, there was already up to 25% of activated CD4 T cells and 20% of activated CD8 T cells after 48 h of incubation.

FIG. 5. Redirected T cell killing of ROR1-positive ovarian cancer target cells with different level of surface ROR1: high expressing PA-1 (A),medium expressing COLO-704 (B) and OVCAR-5 (C), and low expressing SK-OV-3 (D). Effector cells to tumor cells (E:T) ratios of 10 PBMCs : I target cell. Specific cytotoxicity of target cells (tumor lysis) induced by anti-ROR1/anti-CD3 TCB antibodies was measured by LDH release (48h culture). There was a concentration dependent response with increasing concentrations from 0.5 04 to 50 nM. ROR1 Mab1-TCB (squares) induced a concentration-dependent increase in tumor cell lysis of ROR1 high-expressing PA-1 ovarian cancer cells (A), ROR1 medium-expressing COLO-704 (B) and OVCAR-5 (C) ovarian cancer cells and ROR1 low-expressing SK-OV-3 ovarian cancer cells (D). In contrast, control-TCB (A, B, C, circles) which only binds to CD3 did not induce tumor cell lysis at clinically relevant concentrations up to 10 nM). Representative experiments shown.

### DETAILED DESCRIPTION OF THE INVENTION

The term “ROR1” as used herein relates to human ROR1 (synonyms: tyrosine-protein kinase transmembrane receptor ROR1, neurotrophic tyrosine kinase, receptor-related 1, UniProtKB Q01973) which is a tyrosine-protein kinase receptor. The extracellular domain of ROR1 consists according to UniProt of amino acids 30-406. The term “antibody against ROR1, anti ROR1 antibody or ROR1 Mab” as used herein relates to an antibody specifically binding to human ROR1 . The antibody binds specifically to the extracellular domain of ROR1 (amino acids MI-V406 of SEQ ID NO:1). The antibody binds specifically to fragments of the extracellular domain, which are the Ig-like C2-type domain (amino acids Q73-V139 of SEQ ID NO: I), the frizzled domain (amino acids E165-1299 of SEQ ID NO: I), or the kringle domain (amino acids K3 I 2-C391 of SEQ ID NO:1). These fragments are mentioned in 502005100605. It is further preferred that the antibody binds specifically to the extracellular domain fragment WNISSELNKDSYLTL (SEQ ID NO.18) of ROR1. This fragment is mentioned in Daneshmanesh AH et al., int. J. Cancer, 123 (2008) 1190-1195,

The term “CD3ε or CD3” as used herein relates to human CD3ε described under UniProt P07766 (CD3E^{—}HUMAN). The term “antibody against CD3, anti CD3 antibody” relates to an antibody binding to CD3ε. Preferably the antibody comprises a variable domain VH comprising the heavy chain CDRs of SEQ ID NO: 12, 13 and 14 as respectively heavy chain CDR1, CDR2 and CDR3 and a variable domain VL comprising the light chain CDRs of SEQ ID NO: 15, 16 and 17 as respectively light chain CDR1, CDR2 and CDR3. Preferably the antibody comprises the variable domains of SEQ NO:10 (VH) and SEQ NO:11 (VL). Preferably the antibody comprises a variable domain VH comprising the heavy chain CDRs of SEQ ID NO: 23, 24 and 25 as respectively heavy chain CDR1, CDR2 and CDR3 and a variable domain VL comprising the light chain CDRs of SEQ ID NO: 26, 27 and 28 as respectively light chain CDR1, CDR2 and CDR3 Preferably the antibody comprises the variable domains of SEQ IL) NO:21 (VH) and SEQ ID NO:22 (VL).

Instead to CD3, the bispecific antibody used according to the invention can bind specifically to a different target which is also capable of recruiting the activity of a human immune effector cell by specifically binding to an effector antigen located on the human immune effector cell..

“Specifically binding to CD3 or ROR1” refer to an antibody that is capable of binding CD3 or ROR1 (the targets) with sufficient affinity such that the antibody is useful as a therapeutic agent in targeting CI)3 or ROR1. In some embodiments, the extent of binding of an anti-CD3 or ROR1 antibody to an unrelated, non-CD3 or non-ROR1 protein is about 10-fold preferably>100-fold less than the binding of the antibody to CD3 or ROR1 as measured, e.g., by surface plasmon resonance (SPR) e.g. Biacore®, enzyme-linked immunosorbent (ELISA) or flow cytometry (FACS). Preferably the antibody that binds to CD3 or ROR1 has a dissociation constant (1(d) of 10⁻⁸ M or less, preferably from 10⁻⁸ M to 10⁻¹³ M, preferably from 10⁻⁹ M to 10⁻¹³ M. Preferably the bispecific antibody according to the invention binds to an epitope of ROR1 that is conserved among ROR1 from different species and/or an epitope of CD3 that is conserved among CD3 from different species, preferably among human and cynomolgus. “Bispecific antibody specifically binding to CD3 and ROR1” or “antibody according to the invention” refers to a respective definition for binding to both targets. An antibody specifically binding to ROR1 (or CD3 or ROR1 and CD3) does not bind to other human antigens. Therefore in an ELISA, OD values for such unrelated targets will be equal or lower to that of the limit of detection of the specific assay, preferably equal or lower as 1.5 pM, or equal or lower to OD values of control samples without plate-bound-ROR1 or with untransfected HEK293 cells.

Antibodies according to the invention are analyzed by ELISA for binding to human ROR1 using plate-bound ROR1. For this assay, an amount of plate-bound ROR1 preferably or 1.5 nM and concentration(s) preferably ranging from 1 pM to 200 nM of anti-ROR1 antibody are used. An antibody according to the invention for which its ROR1 binding is at least 20% higher than the OD values of the control samples without plate-bound ROR1 or with untransfected HEK293 cells according to the invention is an antibody “binding to human ROR1 in an ELISA assay”.

The term “antibody according to the invention which does not internalize” as used herein means a bispecific antibody according to the invention with MFI reduction properties characterized in that in a cell based assay at 37° C. during 2 firs, using ROR1 -positive B-CLL cells, and used at an antibody concentration of 1 nM, whereby not internalize means, that the mean fluorescence intensity (MFI), as detected by flow cytometry, upon binding to ROR1 -positive cells measured at time 0 is not reduced more than. 50%, preferably not more than 30% by internalization when re-measured after a 2 hr-incubation at 37° C. The bispecific antibody according to the invention does not internalize in ROR1-positive B-CLL cells, therefore the binding of the said anti-ROR1 antibody to ROR1 -positive B-CLL cells is not reduced more than 50%, preferably not more than 30%, when the said antibody is incubated at 37° C. for 2 h in such cell based assay as described herein.

It is also preferred, that a bispecific antibody according to the invention shows in a cell based assay at 37° C. during 2 hrs, using ROR1 -positive B-CLL cells, and at an antibody concentration of 1 nM, a decrease in the mean fluorescence intensity by internalization from time 0 to 2 Ins at 37° C. (AMFI), as measured by flow cytometry is between 120% to 0%, preferably from 100% to 0%, of the ΔMFI of an anti-ROR bivalent antibody of human IgG I kappa (κ) type comprising as light chain variable domain (VL) the sequence of SEQ ID NO.2 and as variable heavy chain domain (VH) the sequence of SEQ ID NO.6, in the same concentration and experimental conditions.

For a therapy using a T cell bispecific antibody comprising an anti-ROR1 antibody, it is preferred that the antibody does not internalize as defined above for facilitating a stable immune synapse between the tumor cell and the T cell and effective T cell-mediated redirected cytotoxicity.

The term “reduction of mean fluorescence intensity” (ΔMFI) reflecting the internalization of the said anti-ROR1 antibody to ROR1 -positive cells” or “MFI reduction” as used herein refers to the percentage of MFI reduction as calculated for each ROR1 antibodies relative to the unspecific human IgG control (MFI background) and ROR1 antibodies maintained on ice (MFIₘₐₓ) by using the formula ΔMFI=100−100 X [(MFIₑₓₚₑᵣᵢₘₑₙₜₐₗ−MFI_{background})/(MFIₘₐₓ−MFI_{background})]. MFIₑₓₚₑᵣᵢₘₑₙₜₐₗ is the MFI measured with said. ROR1 antibody after 2h incubation at 37° C. An MFI reduction which is at least 75% blocked and reversed by 10 μM endocytosis inhibitor phenylarsine oxide is for example caused by antibody internalization while an MFI reduction which is not blocked by phenylarsine oxide is caused by antibody dissociation. Internalizing anti-ROR1 antibodies are known in the state of the art (Baskar et al., Clin, Cancer Res,, 14(2): 396-404 (2008)).

Preferably the bispecific antibody according to the invention is characterized in that an increase in MFI value at time 2hrs in the presence of 3 μM phenylarsine oxide (PAO) as compared to MFI value at: time 2hrs in the absence of PAO is not more than 30% , preferably not more than 20%, preferably not more that I0° ©, even not more than detection level of the MFI value at time 0.

The term “target” as used herein means either ROR1 or CD3. The term “first target arid second target” means either CD3 as first target and ROR1 as second target or means ROR1 as first target and CD3 as second target.

The term “antibody” as used herein refers to a monoclonal antibody. An antibody consists of two pairs of a “light chain” (LC) and a “heavy chain” (HC) (such light chain (LC) /heavy chain pairs are abbreviated. herein as LC/HC). The light chains and heavy chains of such antibodies are polypeptides consisting of several domains. Each heavy chain comprises a heavy chain variable region (abbreviated herein as HCVR or VH) and a heavy chain constant reaion. The heavy chain constant reaion comprises the heavy chain constant domains CH1 , CH2 and. CH3 (antibody classes IgA, IgD, and IgG) and optionally the heavy chain constant domain CH4 (antibody classes IgE and I(441). Each light chain comprises a light chain variable domain VL and a light chain constant domain CL. The variable domains VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The “constant domains” of the heavy chain and of the light chain are not involved directly in binding of an antibody to a target, but exhibit various effector functions.

The “light chain of an antibody” as used herein is a polypeptide comprising in N-terminal to C-terminal direction a light chain variable domain (VL), and a light chain constant domain (CL), abbreviated as VL-CL. A “crossover light chain (VH-CL)” as used herein is a light chain wherein the VL domain is replaced by the respective VII domain. ”The “heavy chain of an antibody” as used herein is a polypeptide comprising in N-terminal to C-terminal direction a heavy chain variable domain (VH) and a constant heavy chain domain 1 (CH1). A. “crossover heavy chain (VL-CH1)” as used herein is a heavy chain wherein the VH domain is replaced by the respective VL domain.

There exist several approaches for CH3-modifications to enforce the hetemdim.erization, which are well described e.g. in WO96/27011, WO98/050431, EP 1870459, WO2007/110205, WO2007/147901, WO2009/089004, WO2010/129304, WO2011/90754, WO2011/143545, WO2012058768, WO2013157954, WO2013096291. Typically in all such approaches the first CH3 domain and the second CH3 domains are both engineered in a complementary manner so that each CH3 domain (or the heavy chain comprising it) cannot longer homodimerize with itself but is forced to heterodimerize with the complementary engineered other CH3 domain (so that the first and second CH3 domain heterodimerize and no homodimers between the two first or the two second CH3 domains are formed). These different approaches for improved heavy chain heterodimerization are contemplated. as different alternatives in combination with the heavy-light chain modifications (CH1 and VH exchange/replacement in one binding arm) in the antibodies according to the invention which reduce light chain mispairing.

In one preferred embodiment of the invention (in case the antibody according to the invention comprises CH3 domains in the heavy chains) the CI-I3 domains of said multispecific antibody according to the invention can be altered by the “knob-into-holes” technology which is described in detail with several examples in e.g. WO 96/027011, kidgway1.B., et al.. Protein Eng. 9 (1996) 617-621; and Merchant, A.M. et al., Nat. Biotechnol. 16 (1998) 677-681; WO98/ 050431. In this method the interaction surfaces of the two CH3 domains are altered to increase the heterodimerization of both heavy chains containing these two CH3 domains. Each of the two CH3 domains (of the two heavy chains) can be the “knob”, while the other is the “hole”.

Thus in one embodiment of the invention said antibody according to the invention (comprises a CH3 domain in each heavy chain and) is further characterized in that the first CH3 domain of the first heavy chain of the antibody under a) and the second CH3 domain of the second heavy chain of the antibody under b) each meet at an interface which comprises an original interface between the antibody CH3 domains, wherein said interface is altered to promote the formation of the antibody according to the invention, wherein the alteration is characterized in that:

i) the CH3 domain of one heavy chain is altered, so that within the original interface of the CH3 domain of one heavy chain that meets the original interface of the CH3 domain of the other heavy chain within the antibody according to the invention, an amino acid residue is replaced with an amino acid residue having a larger side chain volume, thereby generating a protuberance within the interface of the CH3 domain of one heavy chain which is positionable in a cavity within the interface of the CH3 domain of the other heavy chain and

ii) the CH3 domain of the other heavy chain is altered, so that within the original interface of the second CH3 domain that meets the original interface of the first CH3 domain within the antibody according to the invention an amino acid residue is replaced with an amino acid residue having a smaller side chain volume, thereby generating a cavity within the interface of the second CH3 domain within which a protuberance within the interface of the first CH3 domain is positionable.

Preferably said amino acid residue having a larger side chain volume is selected from the group consisting of arginine (R), phenylalanine (F), tyrosine (Y), tryptophan (W).

In one aspect of the invention both CH3 domains are further altered by the introduction of cysteine (C) as amino acid in the corresponding positions of each CH3 domain such that a disulfide bridge between both CH3 domains can be formed.

Other techniques for CH3-modifications to enforcing the heterodimerization are contemplated as alternatives of the invention and described e.g. in WO96/27011, WO98/050431, EP1870459, WO2007/110205, WO2007/147901, WO2009/089004, WO2010/129304, WO2011/90754, WO2011/143545, WO2012/058768, WO2013/157954, WO2013/157953, WO2013/096291.

In one embodiment the antibody according to the invention is of IgG2 isotype and the heterodimerization approach described in WO2010/129304 can be used alternatively.

The term “antibody” includes e.g. mouse antibodies, human antibodies, chimeric antibodies, humanized antibodies and genetically engineered antibodies (variant or mutant antibodies) as long as their characteristic properties are retained. Especially preferred are human or humanized antibodies, especially as recombinant human or humanized antibodies. The terms “monoclonal antibody” or “monoclonal antibody composition” as used herein refer to a preparation of antibody molecules of a single amino acid composition.

The term “comprising” in regard to the bispecific antibody as used herein means that the bispecific antibody comprises as CD3 and ROR1 binders only those binders mentioned. Therefore a bispecific antibody according the invention comprising a monovalent anti-ROR1 antibody specifically binding to ROR1, and a monovalent antibody specifically binding to CD3 has in regard to CD3 and ROR1 binding only one 5 binding valence thr CD3 and only one valence for ROR1 and is therefore bivalent. A bispecific antibody according the invention comprising a bivalent anti-ROR1 antibody specifically binding to ROR1, and a monovalent antibody specifically binding to CD3 has in regard to ROR1 binding two binding valences and in regard to CD3 binding one valence and is therefore trivalent. Preferably the monovalent antibody specifically binding to CD3 is covalently linked at its C-terminus to the N-terminus of one variable chain of the antibody specifically binding to ROR1.

A “Fab fragment of an antibody” as used herein is a fragment on an antibody that binds to antigens. A Fab fragment of an antibody consists of two pairs of domains. In a wild-type antibody it is composed of one constant and one variable domain of each of the heavy chain (CH1 and VH) and the light chain (CL and. VL). According to the invention such domain pairs can be, due to a crossover, also VH-CL and VL-CH1. In a wild-type antibody and according to the invention the domain of the heavy and light chain domain pairs of a Fab fragment are not chemically linked together and are therefore not scFvs (single chain variable fragments). “Crossover” according to the invention means that preferably in one Fab the domains NIL and VH are replaced by each other. The term “Fab fragment” also includes parts or all of the hinge region, like Fab’ fragment. As used herein, “F(ab)₂ fragment” refers to a bivalent monospecific antibody fragment preferably with a Fc part.

The term “ROR1 Fab” as used within the invention denotes a Fab fragment of the antibody specifically binding to ROR1. Due to the exchange of either the variable regions or the constant regions in the anti-ROR1 antibody Fab fragment (ROR1 Fab), such ROR1 Fab is referred to as “ ROR1 cross Fab” or “crossover ROR1 Fab fragment” According to the invention the ROR1 Fab is not a ROR1 crossFab. By “connected” is meant that the Fab fragments are preferably linked by peptide bonds, either directly or via one or more peptide linker. The term “CD3 Fab” as used within the invention denotes a Fab fragment of the antibody specifically binding to CD3. The CD3 Fab is linked at its N-terminus the C-terminus of the ROR1 Fab. Due to the exchange of either the variable regions or the constant regions in the CD3 Fab, such CD3 Fab is referred to as “CD3 crossFab” or “crossover CD3 Fab fragment”. According to the invention the CD3 Fab is preferably a crossFab.

The term “peptide linker” as used within the invention denotes a peptide with amino acid sequences, which is preferably of synthetic origin. These peptide linkers according to invention are used to connect one of the Fab fragments to the C-or N-terminus of the other Fab fragment to form a multispecific antibody according to the invention. Preferably said peptide linkers are peptides with an amino acid sequence with a length of at least 5 amino acids, preferably with a length of 5 to 100, more preferably of 10 to 50 amino acids. In one embodiment said peptide linker is (GxS)ri or (GxS)nGtn with G=glycine, S=serine, and (x=3, n=3, 4, 5 or 6, and m=0, 1, 2 or 3) or (x=4,n=2, 3, 4 or 5 and m=0, 1, 2 or 3), preferably x=4 and n=2 or 3, more preferably with x=4, n=2. Additionally, linkers may comprise (a portion of) an immunoglobulin hinge region. In one embodiment said peptide linker is (G₄S)₂ (SEQ ID: NO 19).

There are five types of mammalian antibody heavy chains denoted by the Greek letters; α, δ, ε, γ, and μ (Janeway C A, Jr et al (2001). Immunobiology. 5th ed., Garland Publishing). The type of heavy chain present defines the class of antibody; these chains are found in IgA, IgD, IgE, IgG, and IgM antibodies, respectively (Rhoades R,A, Pflanzer R G (2002). Human Physiology, 4th ed., Thomson Learning). Distinct heavy chains differ in size and composition; a and y contain approximately 450 amino acids, while μ and ε have approximately 550 amino acids. Each heavy chain has two regions, the constant region and the variable region. The constant region is identical in all antibodies of the same isotype, but differs in antibodies of different isotype. Heavy chains ₇, a and 6 have a constant region composed of three constant domains CH1 , CH2. and CH3 (in a line), and a hinge region for added flexibility (Woof J, Burton I) Nat Rev Immunol 4 (2004) 89-99); heavy chains μ and ε have a constant region composed of four constant domains CH1, CH2, CH3, and CH4 (Janeway C A, Jr et al (2001). Immunobiology. 5th ed., Garland Publishing). The variable region of the heavy chain differs in antibodies produced by different B cells, but is the same for all antibodies produced by a single B cell or B cell clone. The variable region of each heavy chain is approximately 110 amino acids long and is composed of a single antibody domain. In mammals there are only two types of light chain, which are called lambda (i) and kappa (K). A light chain has two successive domains: one constant domain CL and one variable domain VL. The approximate length of a light chain is 211 to 217 amino acids.

A “bispecific antibody” used according to the invention can have any appropriate format. Bispecific formats are e.g. disclosed. Konterm.ann RE, mAbs 4;2, (2012) 1-16, Mueller D. and Kontermann RE.BioDnigs (2010) Volume 24, Issue 2, pp 89-98). Such a bispecific antibody can he based on e.g. Fabs, IgGs and IgG-like molecules, diabodies, single-chain FV (scFV)s, DARPins-, tandAbs, DARTs, nanobodies, triple bodies, triple heads, CH3 fusion proteins. A bispecific antibody used according to the invention, which comprises a Fc part, can be of any class (e.g. IgA, IgD, IgE, IgG, and IgM, preferably IgG or IgE), or subclass (e.g., IgG1, IgG2, IgG3, IgG4, IgAl and IgA2, preferably IgG1), whereby both antibodies, from which the bivalent bispecific antibody used according to the invention is derived, have an Fc part of the same subclass(e.g. IgG1, IgG4 and the like, preferably IgG1), preferably of the same allotype (e.g. Caucasian),

A “Fc part of an antibody” is a term well known to the skilled artisan and defined on the basis of papain cleavage of antibodies. The antibodies used according to the invention, which comprise an Fc part, contain as Fc part, preferably a Fc part derived from human origin and preferably all other parts of the human constant regions. The Fc part of an antibody is directly involved in complement activation, C1q binding, C3 activation and Fc receptor binding. While the influence of an antibody on the complement system is dependent on certain conditions, binding to C1q is caused by defined binding sites in the Fc part. Such binding sites are known in the state of the art and described e.g. by Lukas, T J., et al., J. Immunol. 127 (1981) 2555-2560; Brunhouse, R., and Cebra, J. J., MoI. Immunol. 16 (1979) 907-917; Burton, D R., et al., 5 Nature 288 (1980) 338-344; Thominesen, J.E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat, see below). Antibodies of subclass IgG1, IgG2 and IgG3 usually show complement activation, C1q binding and C3. activation, whereas IgG4 do not activate the complement system, do not bind C1q and do not activate C3. Preferably the Fc part is a human Fc part. Preferably the Fc part is a human IgG1 Fc part. Preferably the antibody used according to the invention comprises in the human IgG1 Fc part amino acid substitution of Pro329 with glycine or arginine and/or substitutions L234A and L235A, preferably Pro329 with glycine and substitutions L234A and L235A.

Preferably the bispecific antibody used according to the invention comprising constant heavy regions CH2/CH3 of IgG1 subclass is characterized in comprising the mutations L234A, L235A and P239G (numbering according to Kabat) to avoid FcR and C1q binding and minimizing ADCC/CDC. The advantage is that such an antibody of the invention mediates its tumor cell killing efficacy purely by the powerful mechanism of T-cell redirection/activation. Additional mechanisms of action like effects on complement system and on effector cells expressing FcR are avoided and the risk of side-effects is decreased.

Preferably the antibody used according to the invention comprises as Fc part an Fc variant of a wild-type human IgG Fc region, said Fc variant comprising an amino acid substitution at position Pro329 and at least one further amino acid substitution, wherein the residues are numbered according to the EU index of Kabat, and wherein said antibody exhibits a reduced affinity to the human FcγRIIIA and/or FcγRIIA and/or FcγRI compared to an antibody comprising the wildtype IgG Fc region, and wherein the ADCC induced by said antibody is reduced to at least 20% of the ADCC induced by the antibody comprising a wild-type human IgG Fc region. In a specific embodiment Pro329 of a wild-type human Fc region in the antibody used according to the invention is substituted with glycine or arginine or an amino acid residue large enough to destroy the proline sandwich within the Fc/Fcγ receptor interface, that is formed between the proline329 of the Fc and tryptophane residues Trp 87 and Tip 110 of FcγRIII (Sondennann et al Nature 406, 267-273 (20 Jul. 2000)), In a further aspect of the invention the at least one further amino acid substitution in the Fc variant is S228P, E2331², L234A, L235A, L235E, N297A, N297D, or P33 IS and still in another embodiment said at least one further amino acid substitution is L234A (denotes that leucine 234 is substituted by alanine) and L235A. of the human IgG1 Fc region or S228P and L235E of the human IgG4 Fc region. Such Fc variants are described in detail in WO2012130831,

The constant heavy chain of an antibody used according to the invention is preferably of human IgG1 type and the constant light chain is preferably of human lambda (k) or kappa (K) type, preferably of human kappa (K) type.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention arc those in which the constant region has been modified or changed from that of the original antibody to generate the properties according to the invention, especially in regard to C1q binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See, e.g., Morrison, S.L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, Riechmann, L., et al., Nature 332 (1988) 323-327; and Neuberger, M.S., et al., Nature 314 (1985) 268-270. Particularly preferred CDRs correspond to those representing sequences recognizing the targets noted above for chimeric antibodies. Other forms of “humanized antibodies” encompassed by the present invention arc those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon target challenge (see, e.g., Jakobovits, A., et al., Proc. Natl. Acad. Sci. USA 90 (1993) 2551-2555; JakobovitsN., et al,, Nature 362 (1993) 255-258; Bruggemarm, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J.D., et al., J. Mot Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boemer, P., et al., J. Immunol. 147 (1991) 86-95). As already mentioned for chimeric and humanized antibodies used according to the invention the term “human antibody” as used herein also comprises such antibodies which are modified in the constant region to generate the properties according to the invention, especially in regard to Clq binding and/or FcR binding, e.g. by “class switching” i.e. change or mutation of Fc parts (e.g. from IgG1 to IgG4 and/or IgG1/IgG4 mutation).

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies used according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable domain” (variable domain of a light chain (VL), variable region of a heavy chain (VH)) as used herein denotes each of the pair of light and heavy chains which is involved directly in binding the antibody to the target. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the target binding site. The antibody heavy and light chain CDR3 regions play a particularly important role in the binding specificity/affinity of the antibodies used according to the invention and therefore provide a further object of the invention.

The terms “hyperyaria.ble region” or “target-binding portion of an antibody” when used herein refer to the amino acid residues of an antibody which are responsible for target-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. CDRs on each chain are separated by such framework amino acids. Especially, CDR3 of the heavy chain is the region which contributes most to target binding. CDR and FR regions are determined according to the standard definition of Kabat et al., Sequences of Proteins of Immunological Interest, 5th ed., Public Health Smice, National Institutes of Health, Bethesda, MD (1991).

The term “target” or “target molecule” as used herein are used interchangeable and refer to human ROR1 and human CD3ε.

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of a target that is bound by an antibody.

In general there are two vectors encoding the light chain and heavy chain of said antibody specifically binding to the first target, and further two vectors encoding the light chain and heavy chain of said antibody specifically binding to the second target. One of the two vectors is encoding the respective light chain and the other of the two vectors is encoding the respective heavy chain. However in an alternative method for the preparation of a bispecific antibody used according to the invention, only one first vector encoding the light chain and heavy chain of the antibody specifically binding to the first target and only one second. vector encoding the light chain and heavy chain of the antibody specifically binding to the second target can be used for transforming the host cell.

The term “nucleic acid or nucleic acid molecule”, as used herein, is intended to include DNA molecules and RNA molecules. A nucleic acid molecule may be single-stranded or double-stranded, but preferably is double-stranded DNA.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. it is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

The term “transthnnation” as used herein refers to process of transfer of a vectors/nucleic acid into a host cell. If cells without formidable cell wall barriers are used as host cells, transfection is carried out e.g. by the calcium phosphate precipitation method as described by Graham and Van der Eh. Virology 52 (1978) 546ff. However, other methods for introducing DNA into cells such as by nuclear injection or by protoplast fusion may also be used. If prokaryotic cells or cells which contain substantial cell wall constructions are used, e.g. one method of transfection is calcium treatment using calcium chloride as described by Cohen SN, et al, PNAS 1972, 69 (8): 2110-2114.

Recombinant production of antibodies using transformation is well-known in the state of the art and described, for example, in the review articles of Makrides, S. C, Protein Expr. Purif 17 (1999) 183-202; Geisse, S., et al., Protein Expr. Purif. 8 (1996) 271-282; Kaufman, R J., Mot. Biotechnol. 16 (2000) 151-161.; Werner, R. G., et al.. Arzneimittelforschung 48 (1998) 870-880 as well as in U.S. Pat. Nos. 6,331,415 and 4,816,567.

As used herein, “expression” refers to the process by which a nucleic acid is transcribed into mRNA and/or to the process by which the transcribed mRNA (also referred to as transcript) is subsequently being translated into peptides, polypeptides, or proteins. The transcripts and the encoded polypeptides are collectively referred to as gene product. If the polynucleotide is derived from genomic DNA, expression in a eukaryotic cell may include splicing of the mRNA.

A “vector” is a nucleic acid molecule, in particular self-replicating, which transfers an inserted nucleic acid molecule into and/or between host cells. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. Also included are vectors that provide more than one of the functions as described.

An “expression vector” is a polynucleotide which, when introduced into an appropriate host cell, can be transcribed and translated into a polypeptide. An “expression system” usually refers to a suitable host cell comprised of an expression vector that can function to yield a desired expression product.

The bispecific antibodies used according to the invention are preferably produced by recombinant means. Such methods are widely known in the state of the art and comprise protein expression in prokaryotic and eukaryotic cells with subsequent isolation of the antibody polypeptide and usually purification to a pharmaceutically acceptable purity. For the protein expression, nucleic acids encoding light and heavy chains or fragments thereof are inserted into expression vectors by standard methods. Expression is performed in appropriate prokaryotic or eukaryotic host cells like CHO cells, NSO cells, SP2/0 cells, HEK293 cells, COS cells, yeast, or E.coli cells, arid the antibody is recovered from the cells (supernatant or cells after lysis). The bispecific antibodies may be present in whole cells, in a cell lysate, or in a partially purified or substantially pure form. Purification is performed in order to eliminate other cellular components or other contaminants, e.g. other cellular nucleic acids or proteins, by standard techniques, including alkaline/SDS treatment, column chromatography and others well known in the art. See Ausubel, F., et al., ed., Current Protocols in Molecular Biology, Greene Publishing and Wiley Interscience, New York (1987).

Expression in NSO cells is described by, e.g., Barnes, L. M., et al., Cytotechnology 32 (2000) 109-123; and Barnes, L. M., et al., Biotech. Bioeng. 73 (2001) 261-270. Transient expression is described by, e.g., Durocher, Y., et al., Nucl. Acids, Res. 30 (2002) E9. Cloning of variable domains is described by Orlandi, et al., Proc. Natl. Acad. Sci, USA 86 (1989) 3833-3837; Carter, P., et al., Proc. Natl. Acad. Sci, USA 89 (1992) 4285-4289; and Norderhaug, L., et al., J. Immunol. Methods 204 (1997) 77-87. A preferred transient expression system (HEK293) is described by Schlaeger, E.- J., and Christensen, K., in Cytotechnology 30 (1999) 71-83 and by Schlaeger, E.-J., in J. Immunol. Methods 194 (1996) 191-199.

The control sequences that are suitable for prokaryotes, for example, include a promoter, optionally an operator sequence, and a ribosome binding site. Eukaryotic cells are known to utilize promoters, enhancers and polyadenylation signals.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The bispecific antibodies are suitably separated from the culture medium by conventional immunoglobulin purification procedures such as, for example, protein A-Sepharose, hydroxylapatite chromatography, gel electrophoresis, dialysis, or affinity chromatography. DNA or RNA encoding the monoclonal antibodies is readily isolated and sequenced using conventional procedures. The hybridoma cells can serve as a source of such DNA and RNA. Once isolated, the DNA may be inserted into expression vectors, which are then transfected into host cells such as HEK293 cells, CHO cells, or myeloma cells that do not otherwise produce immunoglobulin protein, to obtain the synthesis of recombinant monoclonal antibodies in the host cells.

Amino acid sequence variants (or mutants) of the bispecific antibody are prepared by introducing appropriate nucleotide changes into the antibody DNA, or by nucleotide synthesis. Such modifications can be performed, however, only in a very limited range, e.g. as described above. For example, the modifications do not alter the above mentioned antibody characteristics such as the IgG isotype and target binding, but may improve the yield of the recombinant production, protein stability or facilitate the purification.

T cell bispecific (TCB) binders have very high concentrationitumor-cell-receptor-occupancy dependent potency in cell killing (e.g. EC₅₀ in in vitro cell killing assays in the sub- or low picomolar range; Dreier et al. Int J Cancer 2002), T-cell bispecific binder (TCB) are given at much lower doses than conventional monospecific antibodies. For example, blinatumomab (CD19×CD3) is given at a continuous intravenous dose of 5 to 15 μg/m²/d.ay (i.e. only 0.035 to 0.105 mg/m.²/week) hr treatment of acute lymphocytic leukemia or 60 μg/m′/day for treatment of Non Hodgkin Lymphoma, and the serum concentrations at these doses are in the range of 0.5 to 4 ng/ml (Klinger et al., Blood 2012; Topp et al., J Clin (Nicol 2011; Goebeler et al. Ann Oncol 2011). Due to the very short elimination half life of blinatumoinab clinical administration is via continuous infusion via pump carried at the patients body. Due to longer elimination half life of the antibodies of this invention it is envisaged that for an antibody used according to the invention subcutaneous administration is possible and preferred in the clinical settings (preferably in the dose range of 0.1 to 10 mg/m²once or twice a week, preferably even lower doses). Even at these low concentrations/doses/receptor occupancies, TCB can cause considerable adverse events (Klinger et al., Blood 2012), Improved pharmacokinetics properties of the antibodies of the invention are one measure to potentially reduce adverse events.

In principle it is possible to produce bispecific antibodies against CDS and ROR1 in all formats known in the state of the art. A wide variety of recombinant bispecific antibody formats have been developed in the recent past, e.g. by fusion of, e.g. an IgG antibody format and single chain domains (see e.g. Kontennann RE, mAbs 4:2, (2012) 1-16). Bispecific antibodies wherein the variable domains VL and VH or the constant domains CL and CH1 are replaced by each other are described in WO2009080251 and WO2009080252. Antibody formats and thrmats of bispecific and multispecific antibodies are also pepbodies (WO200244215)_{;} Novel Antigen Receptor (“NAR”) (WO2003014161), diabody-diabody dimers “TandAbs” (WO2003048209), polyalkylene oxide-modified scFv (U.S. Pat. No. 7,150,872), humanized rabbit antibodies (WO2005016950), synthetic immunoglobulin domains (WO2006072620), covalent diabodies (WO2006113665)_{;} flexibodies (WO2003025018), domain antibodies, dAb (WO2004058822), vaccihody (WO2004076489), antibodies with new world primate framework (WO2007019620), antibody-drug conjugate with cleavable linkers (WO2009117531), IgG4 antibodies with hinge region removed (WO2010063785), bispecific antibodies with IgG4 like CH3 domains (WO2008119353), camelid Antibodies (U.S. Pat. No. 6,838,254), nanobodies (U.S. Pat. No. 7,655,759), CAT diabodies (U.S. Pat. No. 5,837,242), bispecific scFv2 directed against target antigen and CD3 (U.S. Pat. No. 7,235,641),), sIgA plAntibodies (U.S. Pat. No. 6,303,341), minibodies (U.S. Pat. No. 5,837,821), IgNAR (US2009148438), antibodies with modified hinge and Fc regions (US2008227958, US2008018.1890), trifunctional antibodies (U.S. Pat. No. 5,273,743), triomabs (U.S. Pat. No. 6,551,592), troybodies (U.S. Pat. No. 6,294,654).

An antibody used according to the invention can be administered once or twice a week s.c. administration.

A bispecific trivalent antibody used according to the invention has advantages on the potency, predictability for efficacy and safety.

An antibody used according to the invention with bivalency to ROR1 and monovalency to CD3 favors binding to the tumor target ROR1 on malignant cells over CD3c on T cells in circulation and avoids CD3 sink, thus increasing; drug exposure in the tumor,

The following examples, sequence listing and figures are provided to aid the understanding of the present invention, the true scope of which is set forth in the appended claims. it is understood that modifications can he made in the procedures set forth without departing from the spirit of the invention.

Sequence Listing

| SEQ NO: | Name |
|---|---|
| 1 | ROR1 extracellular domain |
| 2 | Mab ROR1 VL |
| 3 | CDR1L |
| 4 | CDR2L |
| 5 | CDR3L |
| 6 | Mab ROR1 VH |
| 7 | CDR1H |
| 8 | CDR2H |
| 9 | CDR3H |
| 10 | Mab CD3 VH (H2C) |
| 11 | Mab CD3 VL (H2C) |
| 12 | CDR1H (H2C) |
| 13 | CDR2H (H2C) |
| 14 | CDR3H (H2C) |
| 15 | CDR1L (H2C) |
| 16 | CDR2L (H2C) |
| 17 | CDR3L (H2C) |
| 18 | Extracellular fragment of ROR1 |
| 19 | Linker |
| 20 | Intracellular fragment of ROR1 |
| 21 | Mab CD3 VH (CH2527) |
| 22 | Mab CD3 VL (CH2527) |
| 23 | CDR1H (CH2527) |
| 24 | CDR2H (CH2527) |
| 25 | CDR3H (CH2527) |
| 26 | CDRL1 (CH2527) |
| 27 | CDRL2 (CH2527) |
| 28 | CDRL3 (CH2527) |
| 29 | ROR1 hum IgG1 HC LALA PG |
| 30 | ROR1 hum IgG1 LC |
| 31 | ROR1 × CD3 VH_CL HC knob LALA PG |
| 32 | ROR1 HC hole LALA PG |
| 33 | CD3 VL_CH1 |
| 34 | ROR1 × CD3 VH_CL |
| 35 | (ROR1)2 × CD3 VH_CL |
| 36 | Fc hole LALA PG |

To make the following anti-ROR1/anti-CD3 TCBs used according to the inventi to respective constructs/sequence IDs as mentioned in the table above are needed:

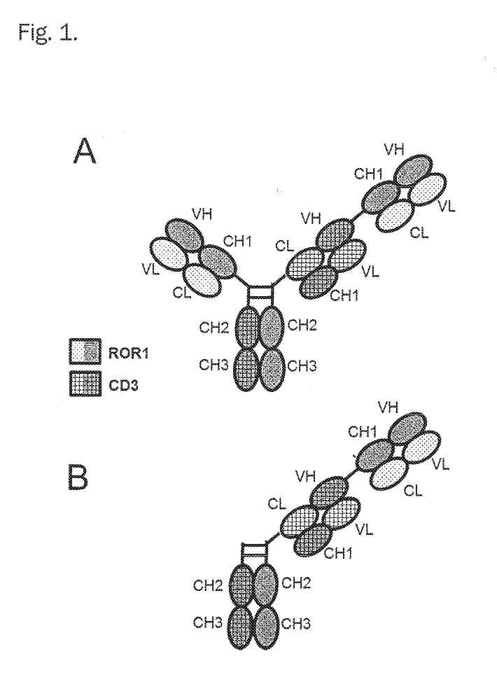

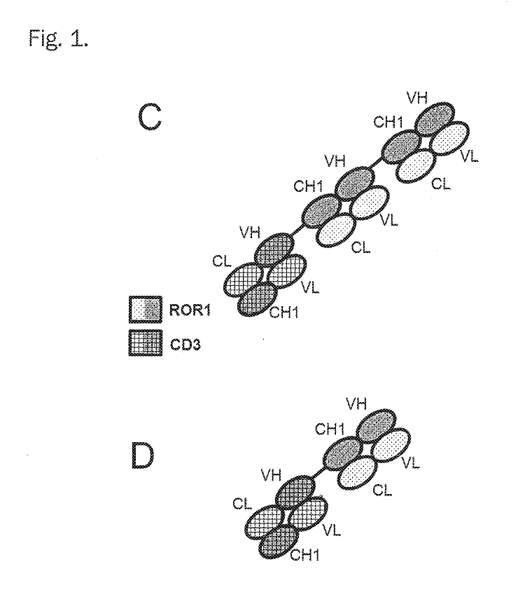

ROR1 -TCB (2+1) Fc-containing: 30 (2×), 31, 32, and 33 (FIG. 1A)

ROR1-TCB (1+1) Fe-containing: 30, 31, 33, and 36 (FIG. 1B)

ROR1-TCB (2+1) non Fc-containing: SEQ ID NO:30 (2×), 33. and 35 (FIG. 1C)

ROR1-TCB (1+1) non Fc-containing SEQ ID NO: 30, 33, and 34 (FIG. 1D)

In the following specific embodiments of the invention are listed:

1. A bispecific antibody specifically binding to the two targets human CD3ε (further named also as “CD3”) and the extracellular domain of human ROR1 (further named also as “ROR1”) for use in the treatment of ovarian cancer.

2. The bispecific antibody according to embodiment 1, characterized in not internalizing in a concentration of InM in primary B-CLL cells at 37° C. during two hours.

3. The bispecific antibody according to any one of embodiment 2, characterized in that the bispecific antibody does not internalize in a cell based assay at 37° C. during 2 hrs, using ROR1 -positive primary B-CLL cells and used at an antibody concentration of I nM. whereby not internalize means, that the mean fluorescence intensity (MFI), as detected by flow cytometry, of said bispecific antibody upon binding to ROR1-positive primary B-CLL cells measured at time 0 is not reduced more than 50%, preferably not more than 30% when re-measured after a 2 hr-incubation at 37° C.

4. The bispecific antibody according to according to any one of embodiments 1 to 3, characterized in 20 consisting of one Fab fragment of an anti-CD3ε antibody (CD3 Fab), one or two Fab fragments of an anti-ROR1 antibody (ROR1 Fab) and no or one Fc fragment.

5. The bispecific antibody according to any one of embodiments 1 to 4, characterized in being bivalent and comprising a monovalent anti-ROR1 antibody specifically binding to ROR1 and a monovalent antibody specifically binding to CD3.

6. The bispecific antibody according to any one of embodiments 1 to 5, characterized in being trivalent and comprising a bivalent anti-ROR1 antibody specifically binding to ROR1, and a monovalent Fab fragment of an antibody specifically binding to CD3.

7, The bispecific antibody according to any one of embodiments 1 to 6, characterized in being selected from the group of the constructs

a) CD3 Fab-ROR1 Fab,

b) CD3 Fab-ROR1 Fab-ROR1 Fab,

c) Fc-CD3 Fab-ROR1 Fab, and

d) ROR1 Fab-Fc CD3 Fab-ROR1 Fab.

8. The bispecific antibody according to any one of embodiments 1 to 7, characterized in that the construct selected from the group of

a) construct consisting of building blocks SEQ ID NO:30 (2×), 31, 32, and 33,

b) construct consisting of building blocks SEQ ID NO:30, 31, 33, and 36,

c) construet consisting of building blocks SEQ ID NO:30 (2×), 33, and 35,

d) construct consisting of building blocks SEQ ID NO: 30, 33, and 34.

9. The bispecific antibody according to any one of embodiments 1 to 8, characterized in that the anti-CD3c antibody sequences VH and VL within SEQ ID NO: 31, 33, 34, 35, 37, 39 are replaced by the respective VH and VL sequences of SEQ II) NO: 21 and 22.

10. The bispecific antibody according to any one of embodiments 1 to 9, characterized in comprising a Fc domain.

11. The bispecific antibody to any one of embodiments 1 to 10, characterized in comprising

a) the light chain and heavy chain of an antibody specifically binding to one of said targets; and

b) the light chain and heavy chain of an antibody specifically binding to the other one of said targets, wherein the variable domains VL and VH or the constant domains CL and CH1 are replaced by each other.

12. The bispecific antibody according to embodiment 11, characterized in that the variable domains VL and VH or the constant domains CL and CH1 of the anti-CD3 antibody are replaced by each other.

13. The bispecific antibody according to any one of embodiments 1 to 12, characterized in that the antibody portion specifically binding to human CD3ε is characterized in comprising

a) a variable heavy chain domain VH comprising the CDRs of SEQ ID NO: 12, 13 and 14 as respectively heavy chain CDR1, CDR2 and CDR3 and a variable domain VL comprising the CDRs of SEQ ID NO: 15, 16 and 17 as respectively light chain CDR1, CDR2 and CDR3, or

b) a variable heavy chain domain VH comprising the CDRs of SEQ ID NO: 23, 24 and 25 as respectively heavy chain CDR1, CDR2 and CDR3 and a variable domain VL comprising the CDRs of SEQ ID NO: 26, 27 and 28 as respectively light chain CDR1, CDR2 and CDR3.

14. The bispecific antibody according to any one of embodiments 1 to 13, characterized in that the antibody portion specifically binding to human ROR1 is characterized in comprising a variable heavy chain domain VH comprising the CDRs of SEQ ID NO: 7, 8 and 9 as respectively heavy chain CDR1, CDR2 and CDR3 and a variable domain VL comprising the CDRs of SEQ ID NO: 3, 4 and 5 as respectively light chain CDR1, CDR2 and CDR3

15. The bispecific antibody according to embodiment 14 , characterized in that said bispecific antibody comprises in addition a second Fab fragment of said first antibody (“ROR1 -Fab”).

16. The bispecific antibody according to any one of embodiment 1 to 15, characterized in consisting of one Fab fragment of an antibody specifically binding to CD3 (further named also as “CD3-Fab”), and one Fab fragment of an antibody specifically binding to ROR1 (further named also as “ROR1 -Fab(s)”) and a Fc part, wherein the CD3-Fab and the ROR1 -Fab are linked via their C-termini to the hinge region of said Fc 1.5 part and wherein the CD3-Fab comprises crossover.

17. The bispecific antibody according to any one of embodiments 1 to 16, characterized in consisting of one CD3-Fab, and one ROR1 -Fab and a Fc part, wherein the CD3-Fab and the ROR1-Fab are linked via their C-termini to the hinge region of said Fc part and a second ROR1 -Fab, which is linked with its C-terminus to the N-terminus of the CD3-Fab and wherein the CD3-Fab comprises crossover (FIG. 1A).

18. The bispecific antibody according to any one of embodiments 1 to 17, characterized in consisting of ROR1 -Fab-Fc-CD3-Fab-ROR1-Fab, wherein the CD3-Fab comprises CL/CH1 crossover.

19. The bispecific antibody according to any one of embodiments 1 to 18, characterized in consisting of two ROR1-Tabs and a Fc part, wherein the ROR1 -Fabs are linked via their C-termini to the hinge region of said Fc part and a CD3-Fab, which is linked with its C-terminus to the N-terminus of one ROR1-Fab and the CD3-Fab comprises crossover (FIG. 1F).

20. The bispecific antibody according to any one of embodiments 1 to 19, characterized in consisting of one CD3-Fab, which is linked via its C-terminus to the hinge region of said Fc part and a ROR1-Fab, which is linked with its C-terminus to the N-terminus of the CD3-Fab (FIG. 1B).

21. The bispecific antibody according to any one of embodiments 1 to 20, characterized in consisting of one ROR1 -Fab, which is linked via its C-terminus to the hinge region of said Fc part and a CD3-Fab, which is linked with its C-terminus to the N-terminus of the ROR1 -Fab (FIG. 1G),

22. The bispecific antibody according to any one of embodiments 1 to 21, characterized in comprising the CDR sequences of anti-ROR1 antibody MAB1.

23. The bispecific antibody according to any one of embodiments 1 to 22, characterized in comprising the VH and VL sequences of anti-ROR1 antibody MAB1, or an antibody comprising the VH, VL, CH1 , and CL sequences of anti-ROR1 antibody MAB1.

24. The bispecific antibody according to any one of embodiments 1 to 23, characterized in that the antibody portion specifically binding to human CD3, preferably the Fab fragment, is characterized in comprising

a) a variable domain VH comprising the heavy chain CDRs of SEQ ID NO: 12, 13 and 14 as respectively heavy chain CDRI CDR2 and CDR3 and a variable domain VL comprising the light chain CDRs of SEQ 10 ID NO: 15, 16 and 17 as respectively light chain CDR1, CDR2 and CDR3 of the anti CD3ε antibody (CDR MAB CD3 H2C), or

b) a variable domain VH comprising the heavy chain CDRs of SEQ m NO: 23, 24 and 25 as respectively heavy chain CDR1, CDR2 and CDR3 and a variable domain VL comprising the light chain CDRs of SEQ ID NO: 26, 27 and 28 as respectively light chain CDR1, CDR2 and CDR3 of the anti CD3ε antibody (CDR MAB CD3 CH2527) .

25. The bispecific antibody according to any one of embodiments 1 to 24, characterized in that )ody portion specifically binding to human CD3 is characterized in that the variable domains are of

a) SEQ ID NO:10 and 11 (VHVL MAB CD3 H2C), or

b) SEQ ID NO:21 and 22 (VHVL MAB CD3 CH2527).

26. The bispecific antibody according to any one of embodiments 1 to 25, characterized in that the Fab fragment, specifically binding to human ROR1 is characterized in comprising a variable domain VH comprising the heavy chain CDRs CDR1H of SEQ ID NO:7, a CDR2H of SEQ ID NO:8, a CDR3H of SEQ NO: 9 and comprising a variable domain VL comprising the light chain CDRs CDR1L of SEQ ID NO:3, a CDR2L of SEQ ID NO:4, a CDR3L of SEQ ID NO: 5 (CDR MAB1).

27. The bispecific antibody according to any one of embodiments 1 to 26, characterized in that the Fab fragment, specifically binding to human ROR1 is characterized in comprising a VII of SEQ ID NO: 10 and a VL of SEQ ID NO: 11 (VHVL MAB1).

28. The antibody according to embodiment 27, characterized in that in the antibody portion specifically binding to human CD3ε

a) the variable domain VH is replaced by a variable domain VH comprising the heavy chain CDRs of SEQ ID NO: 12, 13 and 14 as respectively heavy chain CDR1, CDR2 and CDR3 and the variable domain VL is replaced by a variable domain VL comprising the light chain CDRs of SEQ ID NO: 15, 16 and 17 as respectively light chain CDR1, CDR2 and CDR3 of the anti CD3ε antibody, or

b) the variable domain VH is replaced by a variable domain VH comprising the heavy chain CDRs of SEQ ID NO: 23, 24 and 25 as respectively heavy chain CDR1, CDR2 and CDR3 and the variable domain VL is replaced by a variable domain VL comprising the light chain CDRs of SEQ ID NO: 26, 27 and 28 as respectively light chain CDR1, CDR2 and CDR3 of the anti CD3ε antibody.

29. The antibody according to any one of embodiments 1 to 28, characterized in that the CH3 domain of one heavy chain and the CH3 domain of the other heavy chain each meet at an interface which comprises an original interface between the antibody CH3 domains; wherein said interface is altered to promote the formation of the hispecific antibody, wherein the alteration is characterized in that:

a) the CH3 domain of one heavy chain is altered, so that within the original interface the CH3 domain of one heavy chain that meets the original interface of the CH3 domain of the other heavy chain within the bispecific antibody, an amino acid residue is replaced with an amino acid residue having a larger side chain volume, thereby generating a protuberance within the interface of the CH3 domain of one heavy chain which is positionable in a cavity within the interface, of the (H3 domain of the other heavy chain and

b) the CH3 domain of the other heavy chain is altered, so that within the original interface of the second CH3 domain that meets the original interface of the first CH3 domain within the bispccific antibody an amino acid residue is replaced with an amino acid residue having a smaller side chain volume, thereby generating a cavity within the interface of the second CH3 domain within which a protuberance within the interface of the first CH3 domain is positionable.

30. The antibody according to any one of embodiments 1 to 29, characterized in comprising in the human IgG1 Fc part amino acid substitution of Pro329 with glycine and/or substitutions L234A and L235A.

31. The antibody according to embodiment 30, characterized in being of construct ROR1 Fab-Fe-CD3 Fab-ROR1 Fab and comprising CL/CH1 crossover within the Fab fragment of the anti-CD3 antibody.

32. The antibody according to embodiment 30 or 31., characterized in being of construct ROR1 Fab-Fc-CD3 Fab-ROR1 Fab and comprising a human IgG 1 Fc part with amino acid substitution of Pro329 with glycine and substitutions Leu234 with alanine, and. Leu235 with alanine.

33. The antibody according to any one of embodiments 1 to 32, characterized in specifically binding to the two targets human CD3ε (CD3) and the extracellular domain of human ROR1 (ROR1), characterized in not internalizing in a concentration of InNT in primary B-CLL cells at 37° C. during two hours.

34. The antibody according to any one of embodiments 1 to 33, characterized in specifically binding to the two targets human CD3ε (CD3) and the extracellular domain of human ROR1 (ROR1), characterized in that the bispecific antibody does not internalize in a cell based assay at 37° C. during 2 hrs, using ROR1-positive primary B-CLL cells and used at an antibody concentration of 1 nM, whereby not internalize means, that the mean fluorescence intensity (MFB, as detected by flow cytometry, of said bispecific antibody upon binding to ROR1 -positive primary B-CLL cells measured at time 0 is not reduced more than 50%, preferably not more than 30% when re-measured after a 2hr-incubation at 37° C.

35. The antibody according to embodiments 1 to 34 is characterized by an elimination half-life in mice, preferably cynomolgus monkeys of longer than 12 hours, preferably 3 days or longer.

36. The antibody according to embodiments I to 35 is characterized in showing an EC50 value for binding to ROR1 -positive ovarian cancer cell lines (e.g. PA-1, MCAS, EEO-21, COLO-704, SW-626), preferably PA-1 and/or COLO-704. of 30 nM or lower, preferably an EC50 value of 15 nM and lower.

37. The antibody according to embodiments 1 to 36 is characterized by its capability to induce redirected killing of ROR1 expressing ovarian cancer cells (e.g. PA-1, MCAS, EFO-21, COLO-704, SW-626), preferably PA-1 and/or COLO-704, in the presence of human T cells with an EC50 lower than 10 nM, preferably 1 nM, preferably 0.05 nM, preferably 0.02 nM, preferably 0.002 nM and lower.

38. The antibody according to embodiments 1 to 37 is characterized in that said antibody stored in standard formulation buffer at 37° C. preferably at 40° C., for 10 days, preferably up to 2 weeks, preferably up to 4 weeks, does not result in more than 10% changes (Δ), preferably not more than 5% changes (Δ), in high molecular Iveight (UMW) species and/or low molecular weight (LMW) species and/or monomer content as compared to the said antibody stored in the same formulation buffer at −80° C. for the same period of storage.

39. A pharmaceutical composition comprising an antibody according to any one of embodiments 1 to 38 for use in the treatment of ovarian cancer and a pharmaceutically acceptable excipient.

40. The antibody according to any one of embodiments 1 to 38 or the pharmaceutical composition of embodiment 39 for use as a medicament for use in the treatment of ovarian cancer.

39. An antibody according to any one of embodiments 1 to 38 or the pharmaceutical composition of embodiment 39 for use as a medicament in the treatment of ROR1-positive ovarian cancers.

40. An antibody according to any one of embodiments 1 to 38 or the pharmaceutical composition of embodiment 39 for use as a medicament in the treatment of ovarian cancers.

41. An antibody according to any one of embodiments 1 to 38 or the pharmaceutical composition of embodiment 39 for the treatment of ovarian cancers and for use as a medicament in the treatment of ovarian cancers expressing ROR1.

42. Use of a bispecific antibody according to any one of embodiments 1 to 38 or the pharmaceutical composition of embodiment 39 for the treatment of ovarian cancer in a patient suffering from ovarian cancer.

43. A method of treating ovarian cancer in a patient suffering from ovarian cancer comprising administering to said patient a therapeutically effective amount of a bispecific antibody according to any one of embodiments 1 to 38 or of the pharmaceutical composition of embodiment 39.

Materials & General Methods

Recombinant DNA Techniques

Standard methods ac used to manipulate DNA as described in Sambrook, J. et al., Molecular cloning: A laboratory manual; Cold Spring Harbor Laboratory Press, Cold Spring Harbor, N.Y., 1989. The molecular biological reagents are used according to the manufacturer's instructions. General information regarding the nucleotide sequences of human immunoglobulins light and heavy chains is given in: Kabat, E. A. et al,, (1991) Sequences of Proteins of Immunological Interest, 5th ed.. NIH Publication No 91-3242. Amino acids of antibody chains are numbered and referred to according to Kabat, E.A., et al., Sequences of Proteins of Immunological Interest, 5th ed., Public Health Service, National Institutes of Health, Bethesda, Md., (1991).

Gene Synthesis

a) Desired gene segments are prepared from oligonucleotides made by chemical synthesis. The 600-1800 bp long gene segments, which were flanked by singular restriction endonuclease cleavage sites, are assembled by annealing and ligation of oligonucleotides including PCR amplification and subsequently 15 cloned via the indicated restriction sites e.g. Kpnl/Sad or Ascl/Pacl into a pPCRScript (Stratagene) based pGA4 cloning vector. The DNA sequences of the subcloned gene fragments were confirmed by DNA sequencing. Gene synthesis fragments are ordered according to given specifications at Geneart (Regensburg, Germany).

b) Desired gene segments are required were either generated by PCR using appropriate templates or were synthesized by Geneart AG (Regensburg, Germany) from synthetic oligonucleotides and PCR products by automated gene synthesis. The gene segments flanked by singular restriction endonuclease cleavage sites are cloned into standard expression vectors or into sequencing vectors for further analysis. The plasmid DNA is purified from transformed bacteria using commercially available plastnid purification kits. Plasmid concentration is determined by UV spectroscopy. The DNA sequence of the subcloned gene fragments is confirmed by DNA sequencing, Gene segments are designed with suitable restriction sites to allow sub-cloning into the respective expression vectors. If required, protein coding genes are designed with a 5′-end DNA sequence coding for a leader peptide which targets proteins tbr secretion in eukaryotic cells.

DNA Sequence Determination

DNA sequences are determined by double strand sequencing.

DNA and protein sequence analysis and sequence data management

The Clone Manager (Scientific & Educational Software) software package version 9.2 is used for sequence mapping, analysis, annotation and illustration.

Expression Vectors

a) The fusion genes comprising ⁻the described antibody chains as described below are generated by PCR and/or gene synthesis and assembled with known recombinant methods and techniques by connection of the according nucleic acid segments e.g. using unique restriction sites in the respective vectors. The subcloned nucleic acid sequences are verified by DNA sequencing. For transient transfections larger quantities of the plasmids are prepared by plasmid preparation from transformed E. coli cultures (Nucleobond AX, Macherey-Nagel).

b) For the generation of anti-ROR1 antibody expression vectors, the variable regions of heavy and light chain DNA sequences are subcloned in frame with either the human IgG1 constant heavy chain or the hum IgG1 constant light chain pre-inserted into the respective generic recipient expression vector optimized for expression in mamm.alian cell lines: The antibody expression is driven by a chimeric MPSV promoter comprising a CMV enhancer and a MPSV promoter followed by a 5′ UTR, an intron and a Ig kappa MAR element. The transcription is terminated by a synthetic polyA signal sequence at the 3′ end of the CDS. All vectors carry a 5′-end DNA sequence coding for a leader peptide which targets proteins for secretion in eukaryotic cells. In addition each vector contains an EBV OriP sequence for episomal plasmid replication in EBV EBNA expressing cells.

c) For the generation of ROR1×CD3 bispecific antibody vectors, the IgG1 derived bispecific molecules consist at least of two antigen binding moieties capable of binding specifically to two distinct antigenic determinants CD3 and ROR1. The antigen binding moieties are Fab fragments composed of a heavy and a light chain, each comprising a variable and a constant region. At least one of the Fab fragments was a “Crossfab” fragment, wherein CH1 and CL arc exchanged. The exchange of CH1 and CL within the Fab fragment assures that Fab fragments of different specificity do not have identical domain arrangements. The bispecific molecule design can be monovalent for both antigenic determinants (1+1) or monovalent for CD3 and bivalent for ROR1 where one Fab fragment is fused to the N-terminus of the inner CrossFab (2+1). The bispecific molecule contained an Fc part in order for the molecule to have a loripr, half-life. A schematic representation of the constructs is given in FIG. 1; the preferred sequences of the constructs are shown in SEQ ID NOs 30 to 36. The molecules are produced by co-transfecting HEK293 EBNA cells growing in suspension with the mammalian expression vectors using a polymer. For preparation of 1+1 CrossFab-IgG constructs, cells are transfected with the corresponding expression vectors in a 1:1:1: I ratio (“vector Fc(knoh)” : “vector light chain” : “vector light chain CrossFab” : “vector heavy chain-CrossFab”). For preparation of 2+1 CrossFab-IgG constructs, cells are transfected with the corresponding expression vectors in a 1:2:1:1 ratio (“vector Fc(knob)” : “vector light chain” : “vector light chain CrossFab” : “vector heavy chain-CrossFab”).

Cell Culture Techniques

Standard cell culture techniques are used as described in Current Protocols in Cell Biology (2000), Bonifacino, J. S., Dasso, M., Harford, Lippincott-Schwartz, J. and Yamada, K. M. (eds.), John Wiley & Sons, Inc.

Transient Expression in HEK293 Cells (HEK293-EBNA System)

Bispecific antibodies are expressed by polymer-based transient co-transfection of the respective mammalian expression vectors in HEK293-EBNA cells, which are cultivated in suspension. One day prior to transfection the HEK293-EBNA cells are seeded at 1.5 Mio viable cells/mL in Ex-Cell medium, supplemented with 6 mM of L-Glutamine. For every mL of final production volume 2.0 Mio viable cells are centrifuged (5 minutes at 210×g). The supernatant is aspirated and the cells resuspended in 100 μL of CD CH( )medium. The DNA for every mL of final production volume is prepared by mixing 1 μg of DNA (Ratio heavy chain: modified heavy chain: light chain: modified light chain 1:1:2:1) in 100 μL of CD CHO medium. After addition of 0.27 μL of a polymer solution (1 mg/mL) the mixture is vortexed for 15 seconds and left at room temperature for 10 minutes. After 10 minutes, the resuspended cells and DNA/polymer mixture are put together and then transferred into an appropriate container which is placed in a shaking device (37° C., 5% CO2). After a 3 hours incubation time 800 μL of Ex-Cell Medium, supplemented with 6 mM L-Glutamine, 1.25 mM valproic acid and 12,5% Pepsoy (50 g/L), is added for every ml. of final Production volume. After 24 hours, 70 μL of feed solution is added for every mL of final production volume. After 7 days or when the cell viability is equal or lower than 70%, the cells were separated from the supernatant by centrifugation and sterile filtration. The antibodies are purified by an affinity step and one or two polishing steps, being cation exchange chromatography and size exclusion chromatography. When required, an additional polishing step is used. The recombinant anti-BCMA human antibody and bispecific antibodies are produced in suspension by co-transfecting HEK293-EBNA cells with the mammalian expression vectors using a polymer. The cells are transfected with two or four vectors, depending on the format. For the human IgG1 one plasmid encoded the heavy chain and the other plasmid the light chain. For the bispecific antibodies four plasmids are co-transfected. Two of them encoded the two different heavy chains and the other two encoded the two different light chains. One day prior to transfection the HEK293-EBNA cells are seeded at 1.5 Mio viable cells/mL in F17 Medium, supplemented with 6 mM of L-Giutamine.

Protein Determination

Determination of the antibody concentration is done by measurement of the absorbance at 280 nm, using the theoretical value of the absorbance of a 0.1% solution of the antibody. This value is based on the amino acid sequence and calculated by GPMAW software (Lighthouse data).

SDS-PAGE

The NuPAGE R Pre-Cast gel system (Invitrogen) is used according to the manufacturer's instruction. In particular, 1000 or 4-12% NuPAGEk ⁻Novext Bis-TRIS Pre-Cast gels (pH 6.4) and a NuPAGEk MES (reduced gels, with NuPAGEk Antioxidant running buffer additive) or MOPS (non-reduced gels) running buffer is used.

Protein Purification

By Protein A Affinity Chromatography

For the affinity step the supernatant is loaded on a protein A column (HiTrap Protein A FF , 5 mL, GE Healthcare) equilibrated with 6 CV 20 mM. sodium phosphate. 20 m11,1 sodium citrate, pH 7.5. After a washing step with the same buffer the antibody is eluted from the column by step elution with 20 mM sodium phosphate. 100 mM sodium chloride, 100 mM Glycine, pH 3,0. The fractions with the desired antibody are immediately neutralized by 0.5 M Sodium Phosphate, pH 8.0 (1:10), pooled and concentrated by centrifugation. The concentrate is sterile filtered and processed further by cation exchange chromatography and/or size exclusion chromatography.

By Cation Exchange Chromatography

For the cation exchange chromatography step the concentrated protein is diluted 1:10 with the elution buffer used for the affinity step and loaded onto a cation exchange colutne (Poros 50 HS, Applied Biosystems). After two washing steps with the equilibration buffer and a washing buffer resp. 20 mM sodium phosphate, 20 mM sodium citrate, 20 mM TRIS, pH 5.0 and 20 mM sodium phosphate, 20 mM sodium citrate, 20 TRIS, 100 mM sodium chloride pH 5.0 the protein is eluted with a gradient using 20 mM sodium phosphate, 20 mM sodium citrate, 20 mM TRIS, 100 mM sodium chloride pH 8.5. The fractions containing the desired antibody are pooled, concentrated by centrifugation, sterile filtered and processed further a size exclusion step.

By Analytical Size Exclusion Chromatography

For the size exclusion step the concentrated protein is injected in a XK16/60 HiLoad Superdex 200 column (GE Healthcare), and 20 mM Histidine, 140 mM Sodium Chloride, pH 6.0 with or without Tween20 as formulation buffer. The fractions containing the monomers are pooled, concentrated by centrifugation and sterile filtered into a sterile vial.

Measurement of Purity and Monomer Content

Purity and monomer content of the final protein preparation is determined by CE-SDS (Caliper LabChip GXII system (Caliper Life Sciences)) resp. HPLC (TSKgel G3000 SW XL analytical size exclusion column (Tosoh)) in a 25 irtIVI potassium phosphate, 125 mM Sodium chloride, 200 mM L-arginine monohydrochloride. 0.02% (w/v) Sodium azide, pH 6.7 buffer.

Molecular Weight Confirmation by LC-MS Analyses

Deglycosylation

To confirm homogeneous preparation of the molecules final protein solution of is analyzed by LC-MS analyses. To remove heterogeneity introduced by carbohydrates the constructs are treated with PNGaseF (ProZyme). Therefore the pH of the protein solution is adjusted to pHTO by adding 2 μl 2 M Iris to 20 μg 10 protein with a concentration of 0.5 mg/ml. 0.8 μg PNGaseF is added and incubated for 12 h at 37° C.

LC-MS Analysis-On Line Detection

The LC-MS method is performed on an Agilent HPLC 1200 coupled to a TOT6441 mass spectrometer (Agilent). The chromatographic separation is performed on a Macherey Nagel Polysterene column; RP1000-8 (8 um particle size, 4.6×250 mm; cat. No, 719510). Fluent A is 5% acetonitrile and 0,05% (v/v) formic acid in water, eluent B is 95% acetonitrile, 5% water and 0.05% formic acid. The flow rate is 1 ml/min, the separation is performed at 40° C. and 6 μg (15 μl) of a protein sample obtained with a treatment as described before (table 2).

TABLE 2

| Time (min.) | % B |
|---|---|
| 0.5 | 15 |
| 10 | 60 |
| 12.5 | 100 |
| 14.5 | 100 |
| 14.6 | 15 |
| 16 | 15 |
| 16.1 | 100 |

During the first 4 minutes the eluate is directed into the waste to protect the mass spectrometer from salt contamination. The ESI-source is running with a drying gas flow of 12l/min, a temperature of 350° C. and a nebulizer pressure of 60 psi. The MS spectra are acquired using a fragmentor voltage of 380 V and a mass range 700 to 3200 m/z in positive ion mode using. MS data are acquired by the instrument software from 4 to 17 minutes.

Isolation of Primary Human Pan T Cells from PBMCs

Peripheral blood mononuclear cells (PBMCs) are prepared by Histopaque density centrifugation from enriched lymphocyte preparations (buffy coats) obtained from local blood banks or from fresh blood collected from healthy human donors or ovarian cancer patients. Human PBCMs isolated from ovarian cancer patient blood is collected after infhnned consent is given, in accordance with local ethical committee guidelines and the Declaration of Helsinki. Briefly, blood is diluted with sterile PBS and carefully layered over a Histopaque gradient (Sigma, H8889). After centrifugation for 30 minutes at 450×g at room temperature (brake switched off), part of the plasma above the PBMC containing interphase is discarded. The PBMCs are transferred into new 50 ml Falcon tubes and tubes are filled up with PBS to a total volume of 50 ml. The mixture is centrifuged at room temperature for 10 minutes at 400×g (brake switched on). The supernatant is discarded and the PBMC pellet washed twice with sterile PBS (centrifugation steps at 4° C. for 10 minutes at 350×g). The resulting PBMC population is counted automatically (ViCell) and stored in RPMI1640 medium, containing 10% FCS and 1% Lalanyl-L-glutamine (Biochrom, K0302) at 37° C., 5% C0₂ in the incubator until assay start.

T cell enrichment from PBMCs is performed using the Pan T Cell Isolation Kit II (Miltenyi Biotec #130-091-156), according to the manufacturer's instructions. Briefly, the cell pellets are diluted in 40 μ\tilde{l} cold 20 buffer per 10 million cells (PBS with 0.5% BSA, 2 iuM EDTA, sterile filtered) and incubated with 10 μ\tilde{l} Biotin-Antibody Cocktail per 10 million cells for 10 mm at 4° C. 30 μ\tilde{l} cold buffer and 20 μ\tilde{l} Anti-Biotin magnetic heads per 10 million cells are added, and the mixture incubated for another 15 min at 4° C. Cells are washed by adding 10-20× the current volume and a subsequent centrifugation step at 300×g for 10 min. Up to 100 million cells are resuspended in 500 μ\tilde{l} buffer. Magnetic separation of unlabeled human pan T cells is performed using LS columns (Miltenyi Biotec #130-042-401) according to the manufacturer's instructions. The resulting T cell population is counted automatically (ViCell) and stored in AIM-V medium at 37° C., 5% C0₂ in the incubator until assay start (not longer than 24 h).

Isolation of Primary Human Naive T Cells from PBMCs

Peripheral blood .mc.motruclar cells (PBMCs) are prepared by Histopaque density centrifugation from enriched lymphocyte preparations (huffy coats) obtained from local blood banks or from fresh blood from healthy human donors or ovarian cancer patients. Human PBCMs isolated from ovarian cancer patient blood is collected after informed consent is given, in accordance with local ethical committee guidelines and the Declaration of Helsinki. T-cell enrichment from PBMCs is performed using the Naive CD8⁺ T cell isolation Kit, from Miltenyi Biotec (#130-093-244), according to the manufacturer's instructions, but skipping the last isolation step of CD8⁺ T cells (also see description for the isolation of primary human pan T cells).

### EXAMPLES

### Example 1

### Generation of Anti-ROR1 Antibodies

The protein sequences of the VH and VL regions for an ROR.1 antibody of SEQ ID NOs: 2-9 (MAB I) are described in WO2012/075158. Briefly, oliogonucleotides encoding the above sequences are joined together via PCR to synthesize cDNAs encoding the VH are VL sequences, respectively, of the anti-ROR1 antibody.

For the generation of anti-ROR1 antibody expression vectors, the variable regions of heavy and light chain DNA sequences were subcloned in frame with either the human IgG1 constant heavy chain or the hum IgG1 constant light chain pre-inserted into the respective generic recipient expression vector optimized for expression in mammalian cell lines. The antibody expression was driven by a chimeric MPSV promoter comprising a CMV enhancer and a MPSV promoter followed by a 5′ UTR, an intron and a Ig kappa MAR element. The transcription was terminated by a synthetic polyA signal sequence at the 3′ end of the CDS. All vectors carry a 5′-end DNA sequence coding for a leader peptide which targets proteins for secretion in eukaryotic cells. In addition each vector contained an EBV OriP sequence for episomal plasmid replication in EBV EBNA expressing cells.

ROR1 antibodies were expressed by transient co-transfection of the respective mammalian expression vectors in HEK293-EBNA cells, which were cultivated in suspension, using a polymer. One day prior to transfection the HEK293-EBNA cells were seeded at 1.5 Mio viable cells/mL in Ex-Cell medium, supplemented with 6 mM of L-Glutamine. For every mL of final production volume 2.0 Mio viable cells were centrifuged (5 minutes at 210×g). The supernatant was aspirated and the cells resuspended in 100 μL of CD CHO medium. The DNA for every mL of final production volume was prepared by mixing 1 μg of DNA (Ratio heavy chain: light chain=1:1) in 100 4. of CD CHO medium. After addition of 0.27 μL of solution containing a polymer (1 mg/mL) the mixture was vortexed for 15 seconds and left at room temperature for 10 minutes. After 10 minutes, the resuspended cells and DNA/polymer mixture were put together and then transferred into an appropriate container which was placed in a shaking device (37° C., 5% CO2). After a 3 hours incubation time 800 μL of Ex-Cell Medium, supplemented with 6 inM L-Glutamine, 1.25 mM valproic acid and 12.5% Pepsoy (50 g/L), was added for every mL of final Production volume. After 24 hours, 70 μL of feed solution was added for every mL of final production volume. After 7 days or when the cell viability was equal or lower than 70%, the cells were separated from the supernatant by centrifugation and sterile filtration. The antibodies were purified by an affinity step and one or two polishing steps, being cation exchange chromatography and size exclusion chromatography. When required, an additional polishing step was used. The recombinant anti-ROR1 human antibodies were produced in suspension by co-transfecting HEK293-EBNA cells with the mammalian expression vectors using a polymer. The cells were transfected with two vectors. For the human IgG1 one plasmid encoded. the heavy chain and the other plasmid the light chain. One day prior to transfection the HEK293-EBNA cells were seeded at 1.5 olio viable cells/mL in F17 Medium, supplemented with 6 ITEM of L-Glutamine.

### Example 2

### Human Ovarian Cancer Cell Lines with Different Levels of Expression of ROR1 on the Cell Surface

1) Human ovarian cancer cell line PA-1 derived from ovarian teratocarcinoma is acquired from American Type Culture Collection (ATCC; Cat. No. CRL-1572). PA-1 cell lines are cultured in Eagle's Minimum Essential Medium (MEM) (ATCC, Cat. No. 30-2003) supplemented with 10% fetal bovine serum (heat-inactivated), 2 mM L-glutamne, 1 mM sodium pyruvate, and 1500 mg/L sodium bicarbonate.

2) Human ovarian cancer cell line MCAS derived from mucinous cystadenocarcinoma of the ovary is obtained from the Japanese Collection of Research Bioresources (JCRB; Cat. No, JCRB0240). MCAS cell lines are grown in Eagle's MEM with 20% FBS.

3) Human ovarian cancer cell line EFO-21 derived from ovary cystadenocarcinoma is obtained from Leibniz Institute DSMZ- German Collection of Microorganisms and Cell Cultures (DSMZ; Cat. No. ACC 235). EFO-21 cell lines are cultured in 80% :RPM 1640, 20% heat inactivated fetal bovine serum, 2 mM L-giutainine, ix MEM non-essential amino acids, and 1 mM sodium pyruvate.

4) Human ovarian cancer cell line COLO-704 derived from ovarian adenocarcinoma is obtained from Leibniz Institute DSMZ- German Collection of Microorganisms and Cell Cultures (DSMZ; Cat. No. ACC 198). COLO-704 cell lines are cultured in 90% RPMI 1640 and 10% heat inactivated fetal bovine serum.

5) Human ovarian cancer cell line SW-626 derived from grade III adenocarcinoma is acquired from American Type Culture Collection (ATCC; Cat. No. HTB-78). SW-626 cell lines are cultured in ATCC-formulated Leibovitz's L-15 Medium (Ca. No, 30-2008) and 10% fetal bovine serum.

6) Human ovarian cancer cell line KURAMOCHI derived from undifferentiated carcinoma (ascites) is obtained from the Japanese Collection of Research Bioresources (JCRB; Cat. No. ICR90098). KURAMOCHI cell lines are cultured in RPMI 1640 medium with 10% fetal calf serum.

7) Human ovarian cancer cell line OVSAHO derived from ovarian carcinoma is obtained from the Japanese Collection of Research. Bioresources (JCRB; Cat. No. JCRB1046). OVSAHO cell lines are cultured in RPMI 1640 medium with 10% fetal bovine serum.

8) Human ovarian cancer cell line SNU-119 derived from ovarian cystadenocarcinoma is obtained from the Korean Cell Line Bank (KCLB; Cat. No, 00119). SNU-119 cell lines are cultrured in 52.5% RPMI1640 medium, 40% fetal bovine serum and 7.5% DMSO.

9) Human ovarian cancer cell line COV362 derived from epithelial-endometroid carcinoma is obtained from European Collection of Cell Cultures (ECACC; Cat. No. 07071910). COV362 cell lines are cultrured in DMEM, 2triM glutamine and 10% fetal bovine serum.

10) Human ovarian cancer cell line OVCAR-4 derived from ovary adenocarcinoma is obtained from EZ Biosystems (Cat. No. EZT-OVC4-I). OVCAR-4 cell lines are cultrured in RPMI 1640 medium with 10% fetal bovine serum.

11) Human ovarian cancer cell line COV318 derived from epithelial-endometroid carcinoma is obtained from European Collection of Cell Cultures (ECACC; Cat. No. 07071903). COV318 cell lines are cultured in DMEM, 2 mM glutamine and 10% fetal bovine serum.

12) Human ovarian cancer cell line TYK-nu derived from undifferentiated carcinoma is obtained from the Japanese Collection of Research Bioresources (JCRB; Cat. No, JCRB0234.0), TYK-nu cell lines are cultured in RPMI 1640 medium with 10% fetal calf serum.

13) Human ovarian cancer cell line OVKATE derived from ovarian carcinoma is obtained from the Japanese Collection of Research Bioresources (JCRB; Cat. No. JCRB1044). OVKATE cell lines are cultured in RPMI 1640 medium with 10% fetal calf serum.

14) Human ovarian cancer cell line CAOV-4 derived from adenocarcinoma is acquired from American Type Culture Collection (ATCC; Cat. No. HTB-76). CAC/V-4 cell lines are cultured in ATCC-formulated Leibovitz's L-15 Medium (Cat. No. 30-2008) and 20% fetal bovine serum.

15) Human ovarian cancer cell line OAW28 derived from ovarian carcinoma is obtained from European Collection of Cell Cultures (ECACC; Cat. No. 85101601). OAW28 cell lines are cultured in DMEM, 2 mM. glutamine, sodium pynivate (NaP), 20 IU/l bovine insulin and 10% fetal bovine serum.

16) Human ovarian cancer cell line CAOV-3 derived from adenocarcinoma is acquired from American Type Culture Collection (ATCC; Cat. No. HTB-75). CAOV-3 cell lines are cultured in ATCC-formulated. Dulbecco's Modified Eagle's Medium (Cat. No. 30-2002) and 10% fetal bovine serum.

17) Human ovarian cancer cell line 59M derived from ovarian carcinoma is obtained from European Collection of Cell Cultures (ECACC; Cat. No. 89081802). 59M cell lines are cultured in DMEM, 2 mM glutamine, 1 mM sodium pyruvate (NaP), 20 IU/l bovine insulin and 10% fetal bovine serum.

18) Human ovarian cancer cell. line ONCO-DG-1 derived from ovary adenocarcinoma is obtained from Leihniz Institute DSMZ-German Collection of Microorganisms and Cell Cultures (DSMZ; Cat. No. ACC 507). ONCO-DG-1 cell lines are cultured in 90% RPMI 1640 and 10% heat inactivated fetal bovine serum.

19) Human ovarian cancer cell line NIH: OVCAR-3 derived from ovarian adenocarcinoma is acquired from American Type Culture Collection (ATCC; Cat. No. HTB-161). NIH: OVCAR-3 cell lines are cultured in ATCC-formulated RPMI-1640 Medium (Cat. No. 30-2001), 0.01 mg/mL bovine insulin and 20% fetal bovine serum.

20) Human ovarian cancer cell line ES-2 derived from ovarian clear cell carcinoma is acquired from American Type Culture Collection (ATCC; Cat. No. CRL-1978). ES-2 cell lines are cultured in ATCC-formulated McCoy's 5a Medium Modified (Cat. No. 30-2007) and 10% fetal bovine serum.

21) Human ovarian cancer cell line COV-504 derived from ovarian epithelial-serous carcinoma is obtained from European Collection of Cell Cultures (ECACC; Cat. No. 07071902). COV-504 cell lines are cultured in DMEM, 2 mM glutamine and 10% fetal bovine serum.

22) Human ovarian cancer cell line OV-90 derived from ovarian clear cell carcinoma is acquired from American Type Culture Collection (ATCC; Cat. No, CRL-11732). OV-90 cell lines are cultured in 1:1 mixture of MCDB 105 medium containing a final concentration of 1.5 g/L sodium bicarbonate and Medium 199 containing a final concentration of 2.2 g/L sodium bicarbonate, and 15% fetal bovine serum.

23) Human ovarian cancer cell line RMUG-S derived from ovarian mucinous cystadenocarcinoma is obtained from the Japanese Collection of Research Bioresources (JCRB; Cat. No. 1E050320). RIMUG-S cell lines are cultured in RPMI 1640 medium with 10% fetal calf serum.

24) Human ovarian cancer cell line COV-644 derived from ovarian epithelial-mucinous carcinomais obtained from European Collection of Cell Cultures (ECACC; Cat. No. 07071908). COV-644 cell lines are cultured in DMEM, 2 mM glutamine and 10% fetal bovine serum.

25) Human ovarian cancer cell line SNU-840 derived from ovarian carcinoma is obtained from the Korean Cell Line Bank (KCLB; Cat, No. 00840). SNU-840 cell lines are cultrured in 52.5% RPMI1640 medium , 40% fetal bovine serum and 7.5% DMSO.

26) Human ovarian cancer cell line OVISE derived from ovarian clear cell adenocarcinoma is obtained from the Japanese Collection of Research Bioresources KIM Cat. No. JCRB1043). OVISE cell lines are cultured in RPMI 1640 medium with 10% fetal calf serum.

27) Human ovarian cancer cell line OAW42 derived from ovarian cystadenocarcinoma is obtained from European Collection of Cell Cultures (ECACC; Cat, No. 85073102). OAW42 cell lines are cultured in DMEM, 2 mM glutamine, 1 mM sodium pyruvate (NaP), 20 IU/l bovine insulin and 10% fetal bovine serum.

28) Human ovarian cancer cell line OVTOKO derived from ovarian clear cell adenocarcinoma is obtained from the Japanese Collection of Research Bioresources (JCRB; Cat. No. JCRB1048). OVTOKO cell lines are cultured in RPMI 1640 medium with 10% fetal calf serum.

29) Human ovarian cancer cell line OVMANA derived from ovarian clear cell adenocarcinoma is obtained from the Japanese Collection of Research Bioresources (JCRB; Cat. No. JCRE1045). OVMANA cell lines are cultured in RPMI 1640 medium with 10% fetal calf serum.

30) Human ovarian cancer cell line COV-434 derived from ovarian granulosa tumor is obtained from European Collection of Cell Cultures (ECACC; Cat. No. 07071909). COV-434 cell lines are cultured in DMEM, 2 mM glutamine and 10% fetal bovine serum.

31) Human ovarian cancer cell line OV56 derived from ovarian cystadenocarcinoma is obtained from European Collection of Cell Cultures (ECACC; Cat. No. 96020759). OV56 cell lines are cultured in DMEM:HAMS F12 (1:1), 2 mM Glutamine, 5% Fetal Bovine Serum, 0.5 ug/ml hydrocortisone and 10 ug/ml insulin.

32) Human ovarian cancer cell line SK-OV-3 derived from ovarian carcinoma is acquired from American Type Culture Collection (ATCC; Cat. No. HTB-77). SK-OV-3 cell lines are cultured in ATCC-formulated McCoy's 5a Medium Modified (Cat. No. 30-2007) and 10% fetal bovine serum.

33) Human ovarian cancer cell line A2780 derived from ovarian carcinoma is obtained from European Collection of Cell Cultures (ECACC; Cat. No. 93112519). A2780 cell lines are cultured in RPMI 1640, 2 mM Glutamine and 10% Fetal Bovine Serum

34) Human ovarian cancer cell line IGROV-1 derived from ovary adenocarcinoma is obtained from EZ Biosystems (Cat. No. EZT- IGRO-1). IGROV-1 cell lines are cultnired in RPMI 1640 medium with 10% fetal bovine serum.

35) Human ovarian cancer cell line TOV-21G derived from ovarian carcinoma is acquired from American Type Culture Collection (.TCC; Cat. No. CRL-11730). TOV-21G cell lines are cultured in a 1:1 mixture of MCDB 105 medium containing a final concentration of 1.5 g/L sodium bicarbonate and Medium 199 containing a final concentration of 2.2 g/L sodium bicarbonate and 15% fetal bovine serum.

36) Human ovarian cancer cell line OVCAR-5 derived from ovarian adenocarcinoma was obtained from US National Cancer Institute NCI-60 human cancer cell line panel. OVCAR-5 cell lines were cultured in 90% RPMI 1640 and 10% heat inactivated fetal bovine serum.

### Example 3

### Binding of ROR1 IgG Antibodies to ROR1 -Positive Human Ovarian Cancer Cell Lines (as Detected by Flow Cytometry)

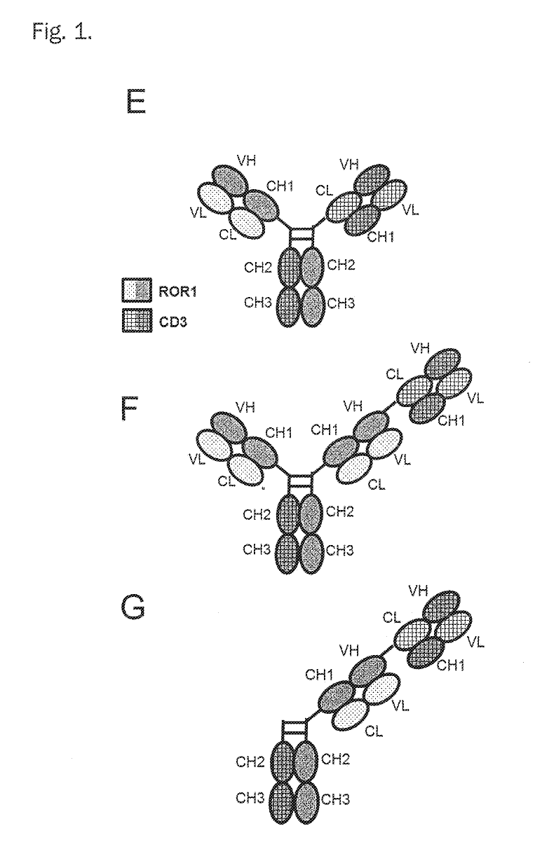

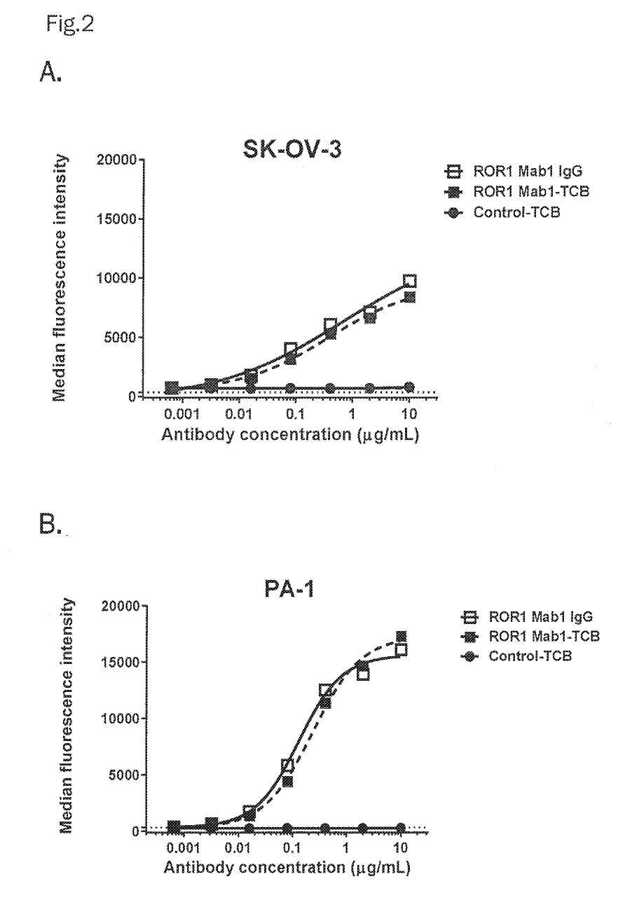

a) The level of expression of ROR1 is measured on human ovarian cancer cell lines by flow cytometry including PA-1, MCAS, EFO-21, COLO-704, SW-626, KURAMOCHI, OVSAHO, SNU-119, COV362, OVCAR-4, COV318, TYK-nu, ON/KATE, CAOV-4, OAW28, CAOV-3, 59M, ONCO-DG-1, OVCAR-3. OVCAR-5, ES-2, COV-504, OV-90, RMUG-S, COV-644, SNU-840, OVISE, OAW42, OVTOKO, OVMANA, COV-434, OV56, SK-OV-3N2780, IGROV-1, and/or TOV-21G. Briefly, cells are harvested, washed, counted for viability, resuspended at 50,000 cells/well of a 96-well round bottom plate and incubated with Alexa488-labeled anti human ROR1 antibody for 30 min at 4° C. All ROR1 and isotype control antibodies are titrated and analyzed in final concentration range between 0.01-100 nM. For samples using non-labelled antibodies, cells are centrifuged (5 min, 350×g), washed with 120 μl/well FACS Stain Buffer (BD Biosciences), resuspended and incubated for an additional 30 min at 4° C. with fluorochrome-conjugated AlexaFluor 647-conjugated AffiniPure F(ab')2 Fragment goat anti-human IgG-Fc Fragment Specific (Jackson Immuno Research Lab; 109-606-008). At the end of incubation time, cells are centrifuged (5 min. at 350 x g), washed twice with FACS buffer, resuspended in 100 u.l FACS buffer and analyzed on a CantoII device running FACS Diva software. Expression of ROR1 is then quantitified as the median fluorescence intensity (MFI) and graphs showing the MFI in function of ROR1 antibody concentrations are plotted. EC50 values are then measured using Prism software (GraphPad). Table 2.1 shows the binding EC50 of Mabl anti-ROR1 antibodies to ROR1 -positive SK-OV-3 and PA-1 ovarian cancer cell lines. The calculated EC50s for binding of ROR1 Mab1 to SK-OV-3 are extrapolated values and may be underestimated. Mabl anti-ROR1 antibodies bind with more potency to PA-1 cell lines (later found to express high level of ROR1) than SK-OV-3 (later found to express low level of ROR1). FIG. 2 shows an increase of MFI on SK-OV-3 cells (A, open squares) and PA-1 cells (B, open triangles) in function of the concentrations of ROR1 Mab2 IgG. Maximum intensity could he reached approximately 3 times more in PA-1 cells vs. SK-OV-3 cells with an antibody concentration of 10 μg/mL.

TABLE 2.1
EC50 values for binding of anti-ROR1 antibodies to
ovarian cancer cell lines

ROR1 Mab1 Binding EC50

| Ovarian cancer cell lines | nM | μg/ml |
|---|---|---|
| SK-OV-3 | ~4.62 | ~0.69 |
| PA-1 | 0.87 | 0.13 |

b) To determine ROR1 antigen copy number on the cell surface of human ovarian cancer cell PA-1 , MCAS, EFO-21, COLO-704, SW-626, KURAMOCIII, OVSAF10, SNU-119, COV362, OVCAR-4, COV318, TYK-nu, OVKATE, CAM-4, OAW28, CAOV-3, 59M, ONCO-DG-1, OVCAR-3, OVCAR-5, ES-2, COV-504, OV-90, RMUG-S, COV-644, SNU-840, OVISE, OAW42, OVTOKO, OVMANA, COY-434, OV56, SK-OV-3, A2780, IGROV-1, and/or TOV-21G. the Qifikit (Dako) method is used. Ovarian tumor cells are once washed with FACS buffer (100 μl/well; 350×g for 5 min) and adjusted to I Mio cells/mi. 50 μl (=0.5 Mio cells) of the cell suspension are transferred into each well of a 96 round bottom well plate, as indicated. Then, 50 ul of mouse anti-human ROR1 IgG antibody (BioLegend #357802) or a mouse IgG2a isotype control antibody (BioLegend #401501) diluted in FACS buffer (PBS, 0.1% BSA) to a final concentration of 25 μg/ml (or at saturation concentrations) are added and staining is performed for 30 min at 4° C. in the dark. Next. 100 μl of the Set-up or Calibration Beads are added in separate wells and the cells, as well as the beads are washed twice with FACS buffer. Cells and beads are resuspended in 25 μl FACS buffer, containing fluorescein conjugated anti-mouse secondary antibody (at saturation concentrations), provided with the Qifikit. Cells and beads are stained for 45 min at 4° C. in the dark. The cells are washed once and all samples are resuspended in 100 μl FACS buffer. Samples are analyzed on a multicolor flow cytometer and installed software (e.g. Canton, device running FACS Diva software or FACSCalibur flow cytometer using the CelIQUEST software).

As shown in Table 2.2, ROR1 antigen copy number/binding sites were measured on five human ovarian cancer cell lines (ES-2, SK-OV-3, OVCAR-5, COLO-704 and PA-1) and expressed at different levels. ES-2 cells did not express any antigen copy of human ROR1 while SKOV-3 cells expressed low level of human ROR1, OVCAR-5 and COLO-704 cells expressed medium level of human ROR1 and PA-1 cells expressed high level of human ROR1.

Based on ROR1 expression results, human ovarian cancer cell lines with high, medium and/or low expression level of ROR1 are selected and used in the redirected T-cell cytotoxicity assay as tumor target cells.

TABLE 2.2
ROR1 antigen copy number/binding sites on human ovarian
cancer cell lines as measured by quantitative flow cytometry

| Human ovarian | ROR1 antigen copy number/ | ROR1 |
|---|---|---|
| cancer cell lines | binding sites | level of expression |
| ES-2 | 0 | Negative |
| SK-OV-3 | 3210 | Low |
| OVCAR-5 | 5034 | Medium |
| Colo704 | 6409 | Medium |
| PA-1 | 14106 | High |

### Example 4

### Generation of Anti-ROR1Anti-CD3 T Cell Bispecific Antibodies

### Example 4.1

### Generation of Anti-CD3 Antibodies

The following protein sequences of the VH and VL regions were used to generate human and cynomolgus monkey cross reactive CD3ε antibodies.

CH2527_VH (SEQ ID NO: 21):
EVQLLESGGGLVQPGGSLRLSCAASGFTFSTYAMNWVRQAPGKGLEWVSR
IRSKYNNYATYYADSVKGRFTISRDDSKNTLYLQMNSLRAEDTAVYYCVR
HGNFGNSYVSWFAYWGQGTLVTVSS
CH2527_VL (SEQ ID NO: 22)
QAVVTQEPSLTVSPGGTVTLTCGSSTGAVTTSNYANWVQEKPGQAFRGLI
GGTNKRAPGTPARFSGSLLGGKAALTLSGAQPEDEAEYYCALWYSNLWVF
GGGTKLTVL

Briefly, oligonucleotides encoding the above sequences were joined together via PCR to synthesize cDNAs encoding the VH and VL sequences, respectively, of the anti-CD3 antibody.

Anti-CD3 antibody CH2527 (SEQ ID NO:21-28) was used to generate the T cell bispecific antibodies which were used in the following examples.

### Example 4.2.

### Generation of Anti-ROR1/Anti-CD3 T Cell Bispecific 1+1 Formats: Bispecific (Fab×(Fab) Antibody Monovalent for ROR1 and Monovalent for CD3

Anti-ROR1/ariti-CD3 T cell bispecific of the 1+1 one-arm format (i.e. bispecific (Fab)×(Fab) antibody monovalent for ROR1 and monovalent for CD3) are produced with the anti-ROR1 antibodies generated from Example 1. eDNAs encoding the full Fabs (heavy chain VH and CH1 domains plus light chain VL and CL domains) of the corresponding anti-ROR1 IgG1 antibodies, as described in Example 1, as well as the anti-CD3 VH and VL cDNAs described in Example 4.1, are used as the starting materials. For each bispecific antibody, four protein chains are involved comprising the heavy and light chains of the corresponding anti-ROR1 antibody and the heavy and light chains of the anti-CD3 antibody described above.

For the generation of ROR1×CD3 bispecific antibody vectors, the IgG1 derived bispecific molecules consist at least of two antigen binding moieties capable of binding specifically to two distinct antigenic determinants CD3 and ROR1. The antigen binding moieties are Fab fragments composed of a heavy and a light chain, each comprising a variable and a constant region. At least one of the Fab fragments is a “Crossfab” fragment, wherein the constant domains of the Fab heavy and light chain are exchanged. The exchange of heavy and light chain constant domains within the Fab fragment assures that Fab fragments of different specificity do not have identical domain arrangements and consequently do not interchange light chains. The bispecific molecule design can be monovalent for bath antigenic determinants (1+1) or monovalent for CD3 and bivalent for ROR1 where one Fab fragment is fused to the N-terminus of the inner CrossFab (2+1). A schematic representation of the constructs is given in FIG. 1. Sequences of the constructs are shown in SEQ ID NOs 30 to 36. The molecules are produced by co-transfecting HEK293 EBNA cells growing in suspension with the mammalian expression vectors using a polymer. For preparation of 1+1 CrossFab-IgG constructs, cells are transfected with the corresponding expression vectors in a 1:1:1:1 ratio (“vector Fe(knob)” : “vector light chain” : “vector light chain CrossFab” : “vector heavy chain-CrossFab”).

To make the following Fe-containing anti-ROR1/anti-CD3 TCBs (1+1), espective constructs/sequence IDs as mentioned in the sequence listing table (Table 1) are needed:

ROR1 -TCB (1+1) Fc-containing: SEQ ID NO:30, 31, 33, and 36 (Fig. IB)

### Example 4.3

### Generation of Anti-ROR1/Anti-CD3 T Cell Bispecific 2+1 Formats: Bispecific (Fab)₂×(Fab) Antibody Bivalent for ROR1 and Monovalent for CD3)

An anti-ROR1/anti-CD3 T cell bispecific antibody with a 2+1 format i.e. bispecific (Fab)2×(Fab) antibody that is bivalent for ROR1 and monovalent for CD3 would have advantages on potency, predictability for efficacy and safety because it would preferentially bind to the tumor target ROR1 and avoid CD3 antibody sink, thus higher probability for drug exposure focused to the tumor,

Anti -ROR1/anti-CD3 T cell bispecific of the 2+1 format (i.e. bispecific (Fab)2×(Fab) antibody bivalent for ROR1 and monovalent for CD3 are produced with the anti-ROR1 antibodies generated in Example 1. cDNAs encoding the full Fabs (heavy chain VH and CH1 domains plus light chain VL and CL domains) of the corresponding anti-ROR1 IgG1 antibodies, as described in Example 1, as well as the anti-CD3 VH and VI. cDNAs described in Example 4.1, are used as the starting materials. For each hispecific antibody, four protein chains are involved comprising the heavy and light chains of the corresponding anti-ROR1 antibody and the heavy and light chains of the anti-CD3 antibody described above.

For the generation of ROR1×CD3 hispecific antibody vectors, the IgG1 derived hispecific molecules consist at least of two antigen binding moieties capable of binding specifically to two distinct antigenic determinants CD3 and ROR1. The antigen binding moieties are Fab fragments composed of a heavy and a light chain, each comprising a variable and a constant region. At least one of the Fab fragments is a “Crossfab” fragment, wherein the constant domains of the Fab heavy and light chain are exchanged. The exchange of heavy and light chain constant domains within the Fab fragment assures that Fab fragments of different specificity do not have identical domain arrangements and consequently do not interchange light chains. The bispecific molecule design can be monovalent for both antigenic determinants (1+1) or monovalent for CD3 and bivalent for ROR1 where one Fab fragment is fused to the N-terminus of the inner CrossFab (2+1). A schematic representation of the constructs is given in FIG. 1; Sequences of the constructs are shown in SEQ NOs 30 to 36. The molecules are produced by co-transfecting HEK293 EBNA cells growing in suspension with the mammalian expression vectors using a polymer. For preparation of 2+1 CrossFab-IgG constructs, cells are transfected with the corresponding expression vectors in a I :2:1:1 ratio (“vector Fc(knob)” : “vector light chain” : “vector light chain CrossFab” ::“vector heavy chain-CrossFab”).

To make the following anti-ROR1/anti-CD3 TCBs (2+1), the respective constructs/sequence IDs as mentioned in the sequence listing table (Table 1) are needed:

ROR1-TCB (2+1) Fe-containing: SEQ ID NO: 30 (2×), 31, 32, and 33 (FIG. 1A)

### Example 4.4

### Production and Purification of Anti-ROR1/Anti-CD3 T Cell Bispecific Antibodies with or without Charge Variants

For the production of the hispecific antibodies, bispecific antibodies are expressed by transient polymer-based co-fransfection of the respective mammalian expression vectors in HEK293-EBNA cells, which are cultivated in suspension. One day prior to transfection the HEK293-EBNA cells are seeded at 1.5 Mio viable cells/m.L in Ex-Cell. medium, supplemented with 6 . mM. of L-Glutamine. For every mL of final production volume 2.0 Mio viable cells are centrifuged (5 minutes at 210×g). The supernatant is aspirated and the cells resuspended in 100 μL of CD CHO medium. The DNA for every mL of final production volume is prepared by mixing 1 μg of DNA (Ratio heavy chain: modified heavy chain: light chain: modified light chain=1: 1:2: 1) in 100 L of CD CHO medium. After addition of 0.27 μL of polymer solution (1 mg/mL) the mixture is vortexed for 15 seconds and left at room temperature for 10 minutes. After 10 minutes, the resuspended cells and DNA/polymer mixture are put together and then transferred into an appropriate container which is placed in a shaking device (37° C., 5% CO₂). After a 3 hours incubation time 800 μL of Ex-Cell Medium, supplemented with 6 mM L-Glutamine, 1.25 mM valproic acid and 12.5% Pepsoy (50 g/L), is added for every mL of final Production volume. After 24 hours, 70 μL of feed solution is added for every mL of final production volume. After 7 days or when the cell viability is equal or lower than 70%, the cells are separated from the supernatant by centrifugation and sterile filtration. The antibodies are purified by an affinity step and one or two polishing steps, being cation exchange chromatography and size exclusion chromatography. When required, an additional polishing step is used.

For the affinity step the supernatant is loaded on a protein A column (HiTrap Protein A FF 5 mL, GE Healthcare) equilibrated with 6 CV 20 mM sodium phosphate, 20 mM sodium citrate, pH 7.5. After a washing step with the same buffer the antibody is eluted from the column by step elution with 20 sodium phosphate, 100 mM sodium chloride, 100 mM Glycine, pH 3.0. The fractions with the desired antibody are immediately neutralized by 0.5 M Sodium Phosphate, pH 8.0 (1:10), pooled and concentrated by centrifugation. The concentrate is sterile filtered and processed further by cation exchange chromatography and/or size exclusion chromatography.

For the cation exchange chromatography step the concentrated protein is diluted 1:10 with the elution buffer used for the affinity step and loaded onto a cation exchange coluine (Porus 50 HS, Applied Biosystems). After two washing steps with the equilibration buffer and a washing buffer resp. 20 mM sodium phosphate, 20 mM sodium citrate, 20 mM TRIS, pH 5.0 and 20 mM sodium phosphate, 20 mM sodium citrate, 20 mM TRIS, 100 mM sodium chloride pH 5.0 the protein is eluted with a gradient using 20 mM sodium phosphate, 20 mM sodium citrate, 20 mM Tills, 100 mM sodium chloride pH 8.5. The fractions containing the desired antibody are pooled, concentrated by centrifugation, sterile filtered and processed further a size exclusion. step.

For the size exclusion step the concentrated protein is injected in a XKI6160 HiLoad Superdex 200 column (GE Healthcare), and 20 mM Histidine, 140 mM Sodium Chloride, pH 6,0 with or without Tween20 as formulation buffer. The fractions containing the monomers are pooled, concentrated by centrifugation and sterile filtered into a sterile vial.

Determination of the antibody concentration is done by measurement of the absorbance at 280 nm, using the theoretical value of the absorbance of a 0.1% solution of the antibody. This value is based on the amino acid sequence and calculated by GPMAW software (Lighthouse data).

Purity and monomer content of the final protein preparation is determined by CE-SDS (Caliper LabChip GMII system (Caliper Life Sciences)) resp. HPLC (TSKgel G3000 SW XL analytical size exclusion column (Tosoh)) in a 25 mM potassium phosphate, 125 mM Sodium chloride, 200 mM L-arginine monohydrochloride, 0.02% (w/v) Sodium azide, pH 6.7 buffer.

To verify the molecular weight of the final protein preparations and confirm the homogeneous preparation of the molecules final protein solution, liquid chromatography-mass spectometry (LC-MS) is used. A deglycosylation step is first performed. To remove heterogeneity introduced by carbohydrates, the constructs are treated with PNGaseF (ProZyme). Therefore, the pH of the protein solution is adjusted to pH7.0 by adding 2 μl 2 M Tris to 20 μg protein with a concentration of 0.5 mg/ml 0.8 μg PNGaseF is added and incubated for 12 h at 37° C. The LC-MS online detection is then performed. LC-MS method is performed on an Agilent HPLC 1200 coupled to a TOT' 6441 mass spectrometer (Agilent). The chromatographic separation is performed on a Macherev Nagel Polysterene column; IRP1000-8 (8 μm particle size, 4.6×250 nun; cat. No. 719510). Eluent A is 5% Jo acetonitrile and 0.05 (.%) (v/v) formic acid in water, eluent B was 95% acetonitrile, 5% water and 0.05 formic acid. The flow rate was 1 ml/min, the separation is performed at 40° C. and 6 μg (15 μl) of a protein sample obtained with a treatment as described before (table 3).

TABLE 3

| Time (min.) | % B |
|---|---|
| 0.5 | 15 |
| 10 | 60 |
| 12.5 | 100 |
| 14.5 | 100 |
| 14.6 | 15 |
| 16 | 15 |
| 16.1 | 100 |

During the first 4 minutes, the eluate is directed into the waste to protect the mass spectrometer from salt contamination. The ESI-source was running with a drying gas flow of 12 l/min, a temperature of 350° C. and a nebulizer pressure of 60 psi. The MS spectra are acquired using a fragmentor voltage of 380 V and a mass range 700 to 3200 m/z in positive ion mode using. MS data are acquired by the instrument software from 4 to 17 minutes.

### Example 5

### Binding of Anti-ROR1/Anti-CD3 T Cell Bispecific Antibodies to Ovarian Cancer Cells and T Cells (as Measured by Flow Cytometry)

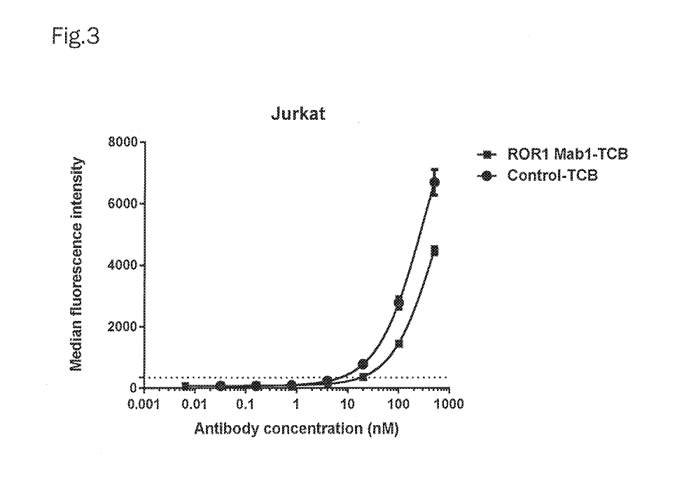

Anti-ROR1/anti-CD3 T cell bispecific antibodies generated in Example 4 are analyzed by flow cytometry for their binding to human ovarian cancer cell lines PA-1, MCAS, EFO-21, COLO-704, and/or SW-626 and human CD3 expressed on human leukemic T cells Jurkat (ATCC TIB-152). Jurkat T cells are cultured in RPMI supplemented with 10% fetal calf serum. Briefly, cultured cells are harvested, counted and cell viability is evaluated using ViCell. Viable cells are then adjusted to 2 ×10⁶ cells per ml in FACS Stain Buffer (BD Biosciences) containing 0.1% BSA. 100 u.1 of this cell suspension are further aliquoted per well into a round-bottom 96-well plate. 30 μl of the Alexa488-labelled anti-ROR¹/ₐnti-CD3 T cell bispecific antibodies or corresponding IgG control were added to the cell-containing wells to obtain final concentrations of 1 nM to 500 nM (Jurkat T cells) or 0.1 nM to 100 nM (human ovarian cancer cells). Anti-ROR1/anti-CD3 T cell bispecific antibodies and control IgG are used at the same molarity, After incubation for 30 min at 4° C., cells are centrifuged (5 min, 350×g), washed twice with 150 μl/well BSA-containing FACS Stain Buffer (BD Biosciences), then cells are fixed using 100 ul BD Fixation buffer per well (#BD Biosciences, 554655) at 4° C. for 20 min, resuspended in 120 μl FACS buffer and analyzed using BD FACS CantoII. Binding of the anti-ROR1/anti-CD3 T cell bispecific antibodies to human ovarian cancer cells and T cells are evaluated and the median fluorescence intensity is determined gated on either human ovarian cancer cells or CD3-expressing Jurkat T cells and plotted in histograms and dot plots. For samples using non-labelled antibodies, cells are centrifuged (5 min, 350×g), washed with 120 μl/well FACS Stain Buffer (BD Biosciences), resuspended and incubated for an additional 30 min at 4° C. with fluorochrome-conjugated AlexaFhior 647-conjugated AffiniPure F(ab')2 Fragment goat anti-human 1gG Fc Fragment Specific (Jackson Immuno Research Lab; 109-606-008). Cells are then washed twice with Stain Buffer (BD Biosciences), fixed using 100 ul BD Fixation buffer per well (#BD Biosciences, 554655) at 4° C. for 20 min, resuspended in 120 μl FACS buffer and analyzed using BD FACS Canton. Median fluorescence intensity for anti-ROR1/anti-CD3 T cell bispecific antibodies in function of antibody concentrations are plotted. EC50 values (denoting the antibody concentration required to reach 50% of the maximal binding) for the binding of anti-ROR1/anti-CD3 antibodies to human ovarian cancer cells are measured using Prism (GraphPad). As depicted in FIG. 2, there was a concentration-dependent binding of ROR1 Mab 1 IgG (open squares) and ROR1 Mab1-TCB (closed squares) SK-OV-3 (A) and on PA-1 human (B) ovarian cancer cell lines as measured by an increase in the median fluorescence intensity signal in function of antibody concentrations. Such positive signals were not observed when the control-TCB binding to CD3 only and not to ROR1 was tested on both SK-OV-3 and PA-1 ovarian cancer cell lines (A and B; closed circles). As shown in FIG. 3, there was a concentration-dependent binding of ROR1 Mab1-TCBcv and control-TCB on Jurkat T cells confirming that both TCB antibodies bind to CD3 on T cells.

### Example 6

### Activation of T Cells upon Engagement of Anti-ROR1/Anti-CD3 T Cell Bispecific Antibodies in the Presence of Ovarian Cancer Cells (Flow Cytometry)

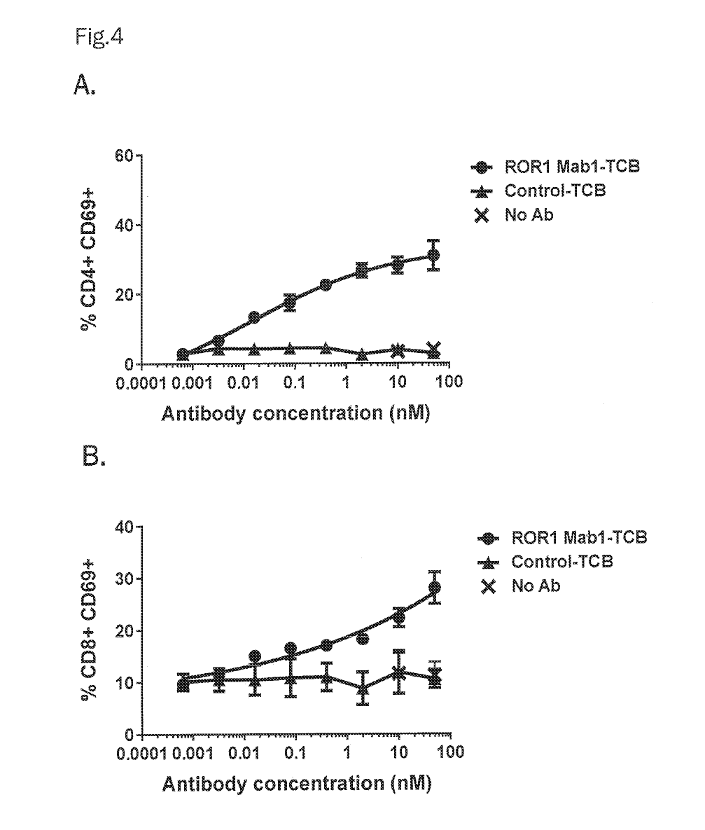

Anti-ROR1/anti-CD3 T cell bispecific antibodies generated in Example 4 are also analyzed by flow cytometry for their potential to induce T-cell activation by evaluating the surface expression of the early activation marker CD69 and/or the late activation marker CD25 on CD4⁺ and CD8⁺ T cells in the presence of ROR1 -positive human ovarian cancer cell lines PA-1, MCAS. EFO-21. COLO-704, and/or SW-626. Briefly, human ovarian cancer target cells are harvested with Trypsin/EDTA, washed, and plated at density of 25,000 cells/well using flat-bottom 96-well plates, Cells are left to adhere overnight. Peripheral blood mononuclear cells (PBMCs) are prepared by Histopaque density centrifugation of enriched lymphocyte preparations (huffy coats) obtained from healthy human donors. Fresh blood is diluted with sterile PBS and layered over Histopaque gradient (Sigma, #H8889). After centrifugation (450×g, 30 minutes, room temperature), the plasma above the PBMC-containing interphase is discarded and. PBMCs transferred in a new falcon tube subsequently filled with 50 ml of PBS. The mixture is centrifuged (400×g, 10 minutes, room temperature), the supernatant discarded and the PBMC pellet washed twice with sterile PBS (centrifugation steps 350×g, 10 minutes). The resulting PBMC population is counted automatically (ViCell) and stored in respective culture medium according to the cell line supplier (see Example 2) at 37° C., 5% CO₂ in a cell incubator until further use (no longer than 24 h). To examine T-cell activation induced by anti-ROR1/anti-CD3 T cell bispecific antibodies, human ovarian cancer cells are exposed to the bispecific antibody at the indicated concentrations (range of 0.1 pM to 200 nM in triplicates). PBMCs are then added to the human ovarian cancer target cells at final effector to target (E:T) ratio of 10:1. T-cell activation is assessed after 24 h to 48 h of incubation at 37° C., 5% CO₂. After the incubation period, cells are collected from the wells, pelleted down by centrifugation (5 min, 350×g) and washed twice with 150 ₁1.1/well of FACS Stain Buffer (BD Biosciences). Surface staining of the effector cells with selected fluorochrome-conjugated antibodies against human CD4 (mouse IgG1,K; clone RPA-T4), CD8 (mouse IgG1,K; clone HIT8a; BT) #555635), CD69 (mouse IgG1 clone L78; BD 4340560) and CD25 (mouse IgG1,K; clone M-A251; BD 4555434) is performed at 4° C. for 30 min, protected from light, in FACS Stain Buffer (BD Biosciences) according to the manufacturer's protocol. Cells are washed twice with 150 μl/well FACS Stain Buffer then fixed using 100 ul BD Fixation buffer per well (#BD Biosciences, 554655) at 4° C. for 20 min, resuspended in 120 μl FACS buffer and analyzed using BD FACS CantoII. The expression of CD69 or CD25 activation markers are determined by measuring the median fluorescence intensity gated on CD4⁺ and CD8⁺ T cell populations as represented in histograms or dot plots. As shown in FIG. 4, ROR1 Mab1 TCB (squares) induced a concentration-dependent increase of CD69 early activation marker which was observed on CD4+ T cells (A) and CD8+ T cells (B) in presence of ROR1-low expressing SK-OV-3 target cells while control-TCB (triangles) did not induce any T-cell activation. At a clinically relevant concentration of I nM of ROR1 Mab1-TCB, there was already up to 25% of activated CD4 I cells and 20% of activated CD8 T cells after 48h of incubation.

### Example 7

### Cell Lysis of Human Ovarian Cancer Cells (LDH Release Assay)

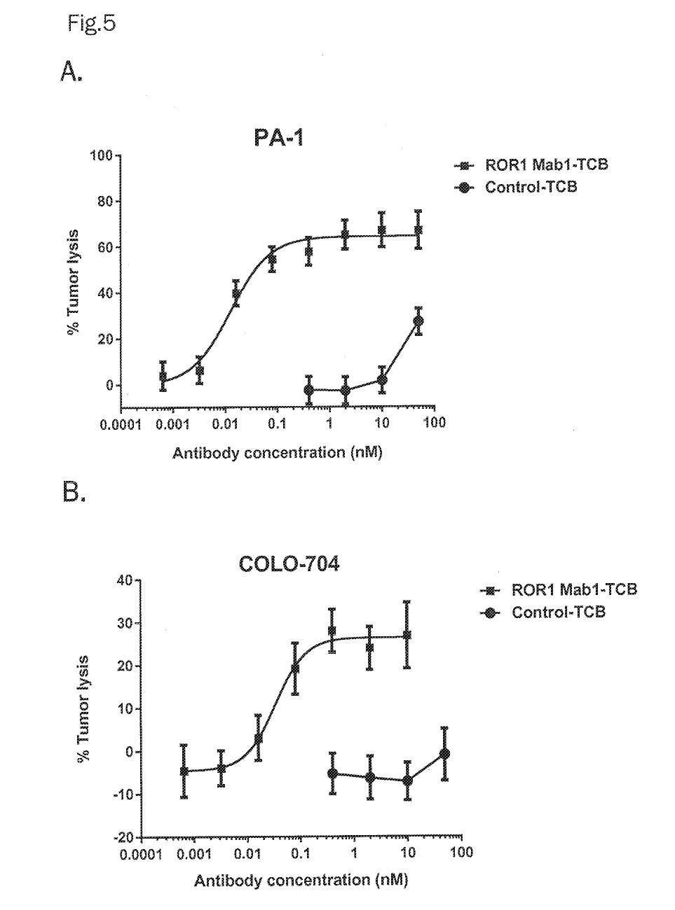

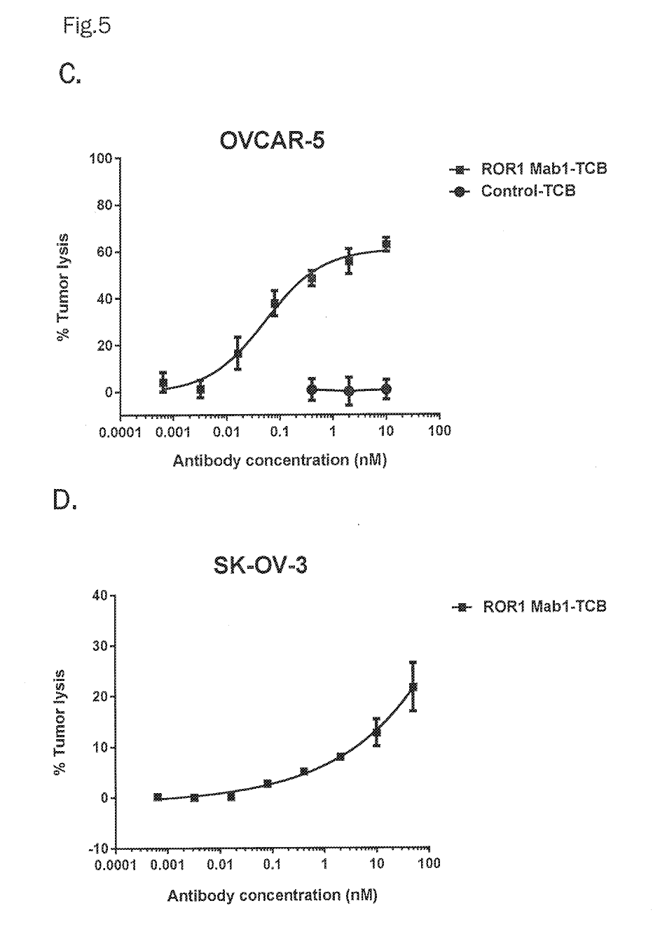

Anti-ROR1/anti-CD3 T cell bispecific antibodies generated in Example 4 are analyzed for induction of T cell-mediated cytotoxicity in human ovarian cancer cells. Human ovarian cancer cell lines PA-1, MCAS, EFO-21, COLO-70^{,}L and/or SW-626. Briefly, human ovarian cancer target cells are harvested with Tr\psin/EDTA, washed, and plated at density of 25,000 cells/well using flat-bottom 96-well plates. Cells are left to adhere overnight. Peripheral blood mononuclear cells (PBMCs) are prepared by Histopaque density centrifugation of enriched lymphocyte preparations (buffy coats) obtained from healthy human donors. Fresh blood is diluted with sterile PBS and layered over Histopaque gradient (Sigma, #H8889). After centrifugation (450×g, 30 minutes, room temperature), the plasma above the PBMC-containing interphase is discarded and PBMCs transferred in anew falcon tube subsequently filled with 50 ml of PBS. The mixture is centrifuged (400×g, 10 minutes, room temperature), the supernatant discarded and the PBMC pellet washed twice with sterile PBS (centrifugation steps 350×g, 10 minutes). The resulting PBMC population is counted automatically (ViCell) and stored in respective culture medium as suggested by the cell line supplier (see Example 2) at 37 ° C., 5% CO₂ in a cell incubator until further use (no longer than 24 h). For the killing assay, the antibody is added at the indicated concentrations (range of 0.1 pM to 200 nM in triplicates). PBMCs are added to the human ovarian cancer target cells at final effector to target (E:T) ratio of 10:1. Target cell killing is assessed after 24 h to 48 h of incubation at 37° C., 5% CO₂ by quantification of LDH released into cell supernatants by apoptotic/necrotic cells (LDH detection kit, Roche Applied Science, #11 644 793 001) fbIlowing the manufacturer's instructions. Maximal lysis of the target cells (=100%) is achieved by incubation of target cells with 1% Triton X-100. Minimal lysis (=-0%) refers to target cells co-incubated with effector cells without bispecific construct. The percentage of LDH release is plotted against the concentrations of anti-ROR1/anti-CD3 I cell hispecific antibodies in concentration-response curves. The IC50 values were measured using Prism software (GraphPad) and determined as the T cell bispecific antibody concentration that results in 50% of LDH release. As shown in FIG. 5, ROR1 Mab1-TCB (squares) induced a concentration-dependent increase in tumor cell lysis of ROR1 high-expressing PA-1 ovarian cancer cells (A), ROR1 medium-expressing COLO-704 (B) and OVCAR-5 (C) ovarian cancer cells and ROR1 low-expressing SK-OV-3 ovarian cancer cells (D). In contrast, control-TCB (A, B, C; circles) which only binds to CD3 did not induce tumor cell lysis at clinically relevant concentrations (i.e. up to 10 nM). Representative experiments shown.

TABLE 9
EC50 values for cell lysis of ovarian cancer cell lines by
anti-ROR1/anti-CD3 T cell bispecific antibodies

ROR1 Mab1-TCB

| Ovarian cancer cell lines | EC50 (pM) | EC50 (ng/mL) |
|---|---|---|
| PA-1 | 12.7 | 2.5 |
| COLO-704 | 34.3 | 6.9 |
| OVCAR-5 | 24.1 | 4.8 |
| SKOV-3 | Not measurable | Not measurable |

1. A bispecific antibody specifically binding to human CD3ε (further named also as “CD3”) and extracellular domain of human ROR1 (further named also as “ROR1”) for use in the treatment of ovarian cancer.

2. The bispecific antibody according to claim 1, characterized in not internalizing in a concentration of inM in primary B-CLL cells at 37° C. during two hours.

3. The bispecific antibody according to claim 1, characterized in that the bispecific antibody does not internalize in a cell based assay at 37° C. during 2 his, using ROR1-positive primary B-CLL cells and used at an antibody concentration of 1 nM, whereby not internalize means that the mean fluorescence intensity (MFI), as detected by flow cytometry, of said bispecific antibody upon binding to ROR1-positive primary B-CLL. cells measured at time 0 is not reduced more than 50%, preferably not more than 30% when re-measured after a 2 hr-incubation at 37° C.

4. The bispecific antibody according to according to claim 1, characterized in consisting of one Fab fragment of an anti-CD3ε antibody (CD3 Fab), one or two Fab fragments of an anti-ROR1 antibody (ROR1 Fab) and no or one Fc fragment.

5. The bispecific antibody according to claim 1, characterized in being bivalent and comprising a monovalent anti-ROR1 antibody specifically binding to ROR1, and a monovalent antibody specifically binding to CD3.

6. The bispecific antibody according to claim 1, characterized in being trivalent and comprising a bivalent anti-ROR1 antibody specifically binding to ROR1, and a monovalent Fab fragment of an antibody specifically binding to CD3.

7. The bispecific antibody according to claim 4, characterized in being selected from the group of the constructs
a) CD3 Fab-ROR1 Fab,
b) CD3 Fab-ROR1 Fab-ROR1 Fab,
c) Fc-CD3 Fab-ROR1 Fab, and
d) ROR1 Fab-Fe-CD3 Fab-ROR1 Fab,

8. The bispecific antibody according to claim 7, characterized in that the construct is selected from the group of
a) construct consisting of building blocks SEQ ID NO:30 (2×), 31, 32, and 33 (FIG. 1A)
b) construct consisting of building blocks SEQ ID NO:30, 31, and 36 (FIG. 1B)
c) construct consisting of building blocks SEQ ID NO:30 (2×), 33, and 35 (FIG. 1C),
d) construct consisting of building blocks SEQ ID NO: 30, 33, and 34 (FIG. 1D).

9. The bispecific antibody according to claim 8. characterized in that anti-CD3ε antibody sequences VH and VL within SEQ ID NO: 31, 33, 34, 35 are replaced by the respective CH1 and CL sequences of SEQ ID NO: 21 and 22.

10. The bispecific antibody according to claim 1, characterized in comprising a Fc domain.

11. The bispecific antibody according to claim 7, characterized in comprising
a) the light chain and heavy chain of an antibody specifically binding to one of said targets; and
b) the light chain and heavy chain of an antibody specifically binding to the other one of said targets, wherein the variable domains VL and VH or the constant domains CL and CH1 are replaced by each other.

12. The bispecific antibody according to claim 11, characterized in that the variable domains VL and VH or the constant domains CL and CH1 of the anti-CD3 antibody are replaced by each other.

13. The bispecific antibody according to claim 7, characterized in that the antibody portion specifically binding to human CD3ε is characterized in comprising
a) a variable heavy chain domain VH comprising the CDRs of SEQ ID NO: 12, 13 and 14 as respectively heavy chain CDR1, CDR2 and CDR3 and a variable domain VL comprising the CDRs of SEQ ID NO: 15, 16 and 17 as respectively light chain CDR1, CDR2 and CDR3, or
b) a variable heavy chain domain VH comprising the CDRs of SEQ ID NO: 23, 24 and 25 as respectively heavy chain CDR1, CDR2 and CDR3 and a variable domain VL comprising the CDRs of SEQ ID NO: 26, 27 and 28 as respectively light chain CDR1, CDR2 and CDR3.

14. The bispecific antibody according to claim 7, characterized in that the antibody portion specifically binding to human ROR1 is characterized in comprising a variable heavy chain domain VH comprising the CDRs of SEQ ID NO: 7, 8 and 9 as respectively heavy chain CDR1, CDR2 and CDR3 and a variable domain VL comprising the CDRs of SEQ ID NO: 3, 4 and 5 as respectively light chain CDR1, CDR2 and CDR3

15. The bispecific antibody according to claim 14, characterized in that said bispecific antibody comprises in addition a second Fab fragment of said first antibody (“ROR1.-Fab”),

16. The bispecific antibody according to claim 1, characterized in comprising the CDR sequences of anti-ROR1 antibody MAB1.

17. The bispecific antibody according to claim 7, characterized in comprising the VH and VL sequences of anti-ROR1 antibody MAB1, or an antibody comprising the VH, VL, CH1, and CL sequences of anti-ROR1 antibody MAB 1.

18. The bispecific antibody according to claim 1, characterized in that the antibody portion specifically binding to human CD3, preferably a Fab fragment, is characterized in comprising
a) a variable domain VH comprising the heavy chain CDRs of SEQ ID NO: 12, 13 and 14 as respectively heavy chain CDR1, CDR2 and CDR3 and a variable domain VL comprising the light chain CDRs of SEQ ID NO: 15, 16 and 17 as respectively light chain CDR1, CDR2 and CDR3 of the anti CD3ε antibody (CDR MAB CD3 H2C), or
b) a variable domain VH comprising the heavy chain CDRs of SEQ ID NO: 23, 24 and 25 as respectively heavy chain CDR1, CDR2 and CDR3 and a variable domain VL comprising the light chain CDRs of SEQ ID NO: 26, 27 and 28 as respectively light chain CDR1, CDR2 and CDR3 of the anti C,D3c antibody (CDR MAB CD3 CH2527).

19. The bispecific antibody according to claim 7, characterized in that the antibody portion specifically binding to human CD3 is characterized in that the variable domains are of
a) SEQ ID NO:10 and 11 (VHVL MAB CD3 H2C), or
b) SEQ ID NO:21 and 22 (VHVL MAB CD3 CH25

20. The bispecific antibody according to claim 1, characterized in that a Fab fragment, specifically binding to human ROR1 is characterized in comprising a variable domain VH comprising the heavy chain CDRs CDR1H of SEQ ID NO:7, a CDR2II of SEQ ID NO:8, a CDR3H of SEQ ID NO: 9 and comprising a variable domain VL comprising the light chain CDRs CDR1L of SEQ ID NO:3, a CDR21_, of SEQ NO:4, a CDR31_, of SEQ ID NO: 5 (CDR MAB1).

21. The bispecific antibody according to claim 1, comprising a Fab fragment, specifically binding to human ROR1 characterized in comprising a VH of SEQ ID NO: 10 and a VL of SEQ ID NO: 11 (VHVL MAB1).

22. The antibody according to claim 21, characterized in that in the antibody portion specifically binding to human CD3ε
a) the variable domain VH is replaced by a variable domain \'H comprising the heavy chain CDRs of SEQ ID NO: 12, 13 and 14 as respectively heavy chain CDR1, CDR2 and. CDR3 and the variable domain VL is replaced by a variable domain VL comprising the light chain CDRs of SEQ ID NO: 15, 16 and 17 as respectively light chain CDR1, CDR2 and CDR3 of the anti CD3ε antibody, or
b) the variable domain VII is replaced by a variable domain VII comprising the heavy chain CDRs of SEQ ID NO: 23, 24 and 25 as respectively heavy chain CDR1, CDR2 and CDR3 and the variable domain VI, is replaced by a variable domain VL comprising the light chain CDRs of SEQ II) NO: 26, 27 and 28 as respectively light chain CDR1, CDR2 and CDR3 of the anti CD3ε antibody.

23. The antibody according to claim 1, characterized in that a first CH3 domain of one heavy chain and a second CH3 domain of the other heavy chain each meet at an interface which comprises an original interface between the antibody CH3 domains; wherein said interface is altered to promote formation of the bispecific antibody, wherein the alteration is characterized in that:
a) the first CH3 domain of one heavy chain is altered, so that within the original interface the first CH3 domain of one heavy chain that meets the original interface of the second CH3 domain of the other heavy chain within the bispecifie antibody, an amino acid residue is replaced with an amino acid residue having a larger side chain volume, thereby generating a protuberance within the interface of the first CH3 domain of one heavy chain which is positionable in a cavity within the interface of the second CH3 domain of the other heavy chain and
b) the second CH3 domain of the other heavy chain is altered, so that within the original interface of the second CH3 domain that meets the original interface of the first CH3 domain within the bi specific antibody an amino acid residue is replaced with an amino acid residue having a smaller side chain volume, thereby generating a cavity within the interface of the second CH3 domain within which a protuberance within the interface of the first CH3 domain is positionable.

24. The antibody according to claim 10, characterized in comprising a human IgG1 Fc part wherein Pro329 is substituted with with glycine and/or substitutions L234A and L235A.

25. The antibody according to claim 24, characterized in being of construct ROR1-Fab-Fc CD3 Fab-ROR1 Fab and comprising CL/CH1 crossover within the Fab fragment of the anti-CD3 antibody.

26. The antibody according to claim 24, characterized in being of construct ROR1 Fab-Fc-CD3 Fab-ROR1 Fab and comprising a human IgG1 Fc part with amino acid substitution of Pro329 with glycine and substitutions Leu234 with alanine and. Leu235 with alanine.

27. The antibody according to claim 26, characterized in specifically binding to the two targets human CD3ε (CD3) and the extracellular domain of human ROR1 (ROR1), characterized in not internalizing in a concentration of 1 nM in primary B-CLL cells at 37° C. during two hours.

28. The antibody according to claim 27, characterized in specifically binding to the two targets human CD3ε (CD3) and the extracellular domain of human ROR1 (ROR1), characterized in that the bispecific antibody does not internalize in a cell based assay at 37° C. during 2 hrs, using ROR1-positive primary B-CLL cells and used at an antibody concentration of 1 nM, whereby not internalize means, that the mean fluorescence intensity (WI), as detected by flow cytometry, of said bispecific antibody upon binding to ROR1-positive primary B-CL1_, cells measured at time 0 is not reduced more than 50%, preferably not more than 30% when re-measured after a 2 hr-incubation at 37° C.

29. The antibody according to claim 28, characterized by an elimination half-life in mice, preferably cynomolgus monkeys of longer than 12 hours, preferably 3 days or longer.

30. The antibody according to claim 29, characterized in showing an EC50 value for binding to ROR1-positive ovarian cancer cell lines PA-1 and/or COLO-704 of 30 nM or lower, preferably an EC50 value of 15 nM and lower.

31. The antibody according to claim 30, characterized by its capability to induce redirected killing of ROR1 expressing ovarian cancer cells PA-1 and/or COLO-704 in the presence of human T cells with an EC50 lower than 10 nM.

32. The antibody according to claim 31, characterized in that said antibody stored in standard formulation buffer at 37° C. for 10 days does not result in more than 10% changes (Δ) in high molecular weight (HIMW) species and/or low molecular weight (JAM) species and/or monomer content as compared to the said antibody stored in the same formulation buffer at −80° C. for the same period.

33. A pharmaceutical composition comprising an antibody according to claim 1 and a pharmaceutically acceptable excipient.

34. (canceled)

35. (canceled)

36. (canceled)

37. (canceled)

38. A method of use of a bi specific antibody according to claim 1 for the treatment of ovarian cancer comprising administering the antibody to a patient suffering from ovarian cancer.

39. A method of treating ovarian cancer in a patient suffering from ovarian cancer comprising administering to said patient a therapeutically effective amount of a bispecific antibody according to claim 1.

40. A method for the treatment of ROR1-positive ovarian cancer comprising administering an antibody according to claim 1 to a subject in need thereof.
